(19) United States
(12) Patent Application Publication
(10) Pub. No.: US 2025/0235850 A1
(43) Pub. Date: Jul. 24, 2025

(54) PREPARING FAST SUPERABSORBENTS WITH ENHANCED ELASTIC BEHAVIOR

(71) Applicant: EVONIK SUPERABSORBER GMBH, Essen (DE)

(72) Inventors: Christoph Loick, Duisburg (DE); Axel Mescher, Hattingen (DE); Martin Dirks, Bochum (DE); Dominik Gartz, Viersen (DE); Scott Smith, Franktown, CO (US); Diedrich Schmidt, Greensboro, NC (US); Axel Pfahler, Krefeld (DE)

(73) Assignee: EVONIK SUPERABSORBER GMBH, Essen (DE)

(21) Appl. No.: 18/703,328

(22) PCT Filed: Oct. 17, 2022
(86) PCT No.: PCT/EP2022/078808

§ 371 Date: Apr. 19, 2024

(30) Foreign Application Priority Data
Oct. 21, 2021 (EP) EP 21203838.4

(51) Int. Cl. B01J 20/30; B01J 20/04; B01J 20/26; B01J 20/28; B01J 20/32

(52) U.S. Cl. CPC B01J 20/3007; B01J 20/043; B01J 20/261; B01J 20/28047; B01J 20/3021; B01J 20/3282; B01J 2220/68

(57) ABSTRACT

Present invention deals with a method for preparing water-absorbing polymers, so called superabsorbents. The invention is based on the finding that extruding hydrogel through a hole plate (13) obeying a complex design rule diminishes negative influence of thixotropy and increases the absorption speed of the superabsorbent made from the hydrogel.

Present invention deals with a method for preparing a water-absorbing polymer and a water-absorbing polymer obtainable thereof.

“Superabsorbent” is a common commercial term referring to polymeric particles capable for absorbing huge amounts of water without releasing it under pressure. Other common terms are “Super-absorbent material” (SAM), “super-absorbent polymers” (SAP) or “absorbent gelling material” (AGM).

When absorbing water, the superabsorbent swells impetuously; the water is caged within its polymeric network so that the water-laden superabsorbent forms a hydrogel. Likewise water, superabsorbents do absorb saline and different kinds body fluids. Due to this capacity, superabsorbents serve as a key ingredient for personal hygiene products like baby diapers, feminine care products and incontinence articles.

Superabsorbents are produced in large industrial scale, the global production is beyond 1 000 000 metric tons per annum.

An introduction into technology of water absorbing polymers is derivable from:
Markus Frank: Superabsorbents. Ullmann's Encyclopedia of Industrial Chemistry. Published Online: 15 Jan. 2003 DOI: 10.1002/14356007.f25_f01

A widespread industrial process for producing superabsorbents comprising the following basic steps:

A monomer mixture is provided. Its essential components are a monomer, a crosslinker and an initiator. Acrylic acid is mostly chosen monomer. As a solvent, water is present within the monomer mixture as well. Other components may be included, for instance co-monomers which will be included in the later polymer or auxiliary components that will not be a part of the later polymer.

The monomer mixture is feed into a reactor and the polymerization is initiated by suitable means depending from the type of initiator. During polymerization, the monomer builds up polymer-chains which are crosslinked by the crosslinker to form a three-dimensional polymer network. Water is caged within the network. Accordingly, the reaction product is a hydrogel.

The hydrogel needs to be disintegrated. This may be done directly within the reaction vessel by means of an agitator (kneading reactor) and/or in one or more separate devices, so called extruders or choppers.

In any case, at the end of the disintegrating process there is at least one extrusion vessel, which is caped with a perforated hole plate, through which the (pre comminuted) hydrogel is extruded. During extrusion, the hydrogel is forced to pass the through holes axially to obtain an extruded hydrogel.

To drive out water from the polymer network, the extruded hydrogel is then dried. Hence, a solid polymer material is obtained. Then, the particle size of the polymer material needs to be adjusted. This is accomplished by grinding and sieving the solid polymer material to obtain a powder with a certain particle size in between 150 μm and 850 μm.

This polymer powder is already capable for absorbing water as a reverse process for the drying step. However, to improve performance, further finishing steps are required:

In particular, the powder is subjected to a surface crosslinking procedure. During surface crosslinking, the density of crosslinks is increased in a surface-near area of the individual particles. Hence, each particle obtains an egg-like core/shell structure with a soft core with a comparable low crosslinking density and a hard shell with a higher degree of crosslinking density. The core/shell structure affect the absorption properties of the SAP significantly.

Beyond absorption properties, other functionalities are adjusted during post processing, for instance odor control, color stability, and favorable storage and conveying properties. This is achieved by adding further ingredients to the polymer powder.

Superabsorbents are usually purchased by manufacturers of personal hygiene products. For purchase decision, the overall performance profile of a superabsorbent is crucial. The required performance profile strongly depends from the type and purpose of the hygiene article. During the last decades, a bunch of performance parameters has been established by the market participants for comparing the suitability of different SAP qualities for an intended purpose. Some of those performance parameters are standardized by independent authorities, others are defined by certain manufacturers of hygiene products for their individual needs only. Actually, both types of performance parameters are of importance within the real-life SAP market. Beside that, the patent literature is jam-packed with parameters which do not have any impact outside a certain patent right at all.

For the present invention, the following performance parameters are relevant:

Storage modulus (G′). The storage modulus describes the ratio of the shear stress (force) to the shear strain (deformation) for elastic solids. Therefore G′ quantifies elasticity. Unit is Pascal. The higher the value, the stiffer the material. The elasticity strongly depends from the water content: A dry SAP is relatively solid, while a water swollen SAP (a hydrogel) shows viscoelastic behavior. Hence, G′ is measured in swollen state. The amount of fluid absorbed by the probe is defined by the related method. As the absorbed amount differs notably between different methods, it is hard to compare values obtained by different methods. Beyond that, viscoelastic properties are recorded with oscillatory measurements influenced by further parameters. The method used herein, is defined below in section “gel bed rheology”. Within (patent) literature, the following terms have been used for describing elasticity of SAP hydrogels instead of storage modulus: elastic modulus, storage elastic modulus, gel strength, gel bed elasticity.

Yield stress (YS). The viscoelasticity of a hydrogel is dependent from time and stress (thixotropy): Subjected to increasing stress or under constant stress over increasing time, the elasticity decreases abruptly. The yield stress is the mechanical load applied in the moment where the elasticity of the hydrogel collapses. Therefore, yield stress locates the point where the gel bed breaks down and the particles move relative to each other resulting in a loss of gel elasticity. The larger the YS is, the more elastic the material behaves, whereas the smaller, the less elastic the material is. Unit of yield stress is Pascal.

A general article dealing with measurement of yield stress is derivable from:
https://cdn.technologynetworks.com/TN/Resources/PDF/WP120416UnderstandYieldStress Meas.pdf

The method to measure yield stress as used herein, is defined below in section “gel bed rheology”. In practical appliance, yield stress is an important value for characterizing reliability: The higher the value, the lower is the risk of viscosity loss due to thixotropic behavior. Lower yield stress means that the negative effect of thixotropy is rather unlikely to be seen.

Free swell rate (FSR). This is a parameter relating to the absorption speed of a SAP without external pressure. It is constructed as a mass speed of absorbed urine per mass unit of SAP. The unit is second, sometimes g/g*s is mentioned. The higher the value, the higher is the absorption speed. This parameter has been established by a major manufacturer of diapers. The test procedure is disclosed in EP 0443627 A2, page 12, lines 24 to 44.

Uptake of 20 g/g (T20). This is a parameter relating to the absorption speed of a SAP without external pressure. It is constructed as the time to absorb a certain amount of urine (20 g) by a certain amount if SAP (1 g). The unit is second. The lower the value, the higher is the absorption speed. The parameter has been established by another major manufacturer of diapers. The test procedure is disclosed in EP 2535697 A1, paragraphs [0027] to [0071].

Centrifugal retention capacity (CRC). One of the oldest performance parameters for SAP. Refers to the capacity for absorbing urine. Unit is g/g or [-]. The higher the value, the higher the capacity. As the test method formally uses a teabag to be filled with SAP, this value has been often referred as “teabag-capacity”. The test method includes centrifugation the loaded probe. However, chosen rotation speed and therefore the centrifugal force G differs from time to time. Hence the value is only reliable with given speed of rotation or G force. The method has been standardized by business organization EDANA as Standard Test WSP 241.2 (05).

Absorbency under a pressure (AUP). Refers to the capacity of absorbing urine when subjected to an external pressure. Unit is g/g or [-]. The higher the value, the higher the capacity. The pressure applied on the probe differs. Some ones use a pressure of 4.83 kPa (=0.7 psi, thus named AUP 0.7), others prefer 2.07 kPa (=0.3 psi, named AUP 0.3 accordingly). Hence, the value is informative only if applied pressure is given. Hererin, AUP has been measured at 4.83 kPa. Sometimes, the AUP value is called “Absorbency under Load (AUL)” as well. The method has been standardized by business organization EDANA as Standard Test WSP 242.2 (05).

As the definition of performance parameters changes over time, comparing certain values is not possible without considering the precise measurement method before its time horizon.

Between individual performance parameters there are known relationships and conflicts of objectives. For instance, measures to enhance AUP may decrease CRC; see Frank cited above, FIG. 7. For this reason, developing a superabsorbent with an improved behavior regarding property X additionally means not to impair property Y. A well-balanced profile of properties optimized for the intended purpose of the SAP is what marked demands.

A common mathematical approach to estimate a balance of conflicting properties is to define an index as an arithmetical function of a multiplicity of property parameters. In the easiest case, the index is defined as the product of two parameters. A well-balance profile of properties represented by the two parameters is fulfilled, if the value of the index is within a certain range.

For the present invention, thixotropic behavior and absorption speed were from interest. In particular, there was a demand for superfast superabsorbents achieving FSR values of at least 0.4 g/g/s and higher. Such superfast superabsorbents are required for ultra-thin diaper constructions: Fast absorption speed together with permeability is the key to replace volumous fluff in diaper constructions Regarding viscoelasticity, it is beneficial if hydrogel shows no thixotropic behavior under load applied during manufacturing, as this favors processing efficiency during production of SAP. In addition to that, gel beds exhibiting their thixotropic character in a loaded diaper are unfavorable: Under long-term external pressure the swollen gel bed may suddenly loose its stability. This may result in leakage or reduced rewet behavior. Thus, a superabsorbent showing no thixotropy in swollen status helps enhancing reliably of the personal hygiene article. A swollen superabsorbent showing no thixotropy under relevant load behave like a NEWTONian fluid. Such vicsoelastic behavior is desired for reasons given above.

For improving absorption speed value FSR, it has been suggested by EP2557095B1 to adjust the mass specific energy applied during committing hydrogel within a certain range. The specific energy is calculated from the electrical power consumption of comminutor's motor and an efficiency factor. While electrical power consumption can be measured with ease, EP2557095B1 fails to give any information how the mechanical efficiency of the comminutor can be determined. In paragraph [0049] it states that the efficiency could be inquired by the manufacturer of gel grinding devices, but this is not the case. Consequently, EP2557095B1 fails to teach the skilled person certain design details of a grinding device resulting in a desired efficiency.

Object of EP2951212B1 is providing a SAP with improved FSR and CRC. The specific energy input during extrusion is capped to 60 kWh/t. The specific energy input shall be influenced by the ratio of length and diameter of the extruder. Some dimensions of the extruder die are given, their impact on FSR is not discussed.

EP3070114A1 aims on improvement of SAP absorbency, namely CRC and AUL. To achieve this, a design rule for chopper dies is disclosed. However, measures influencing absorption speed or thixotropy is not given.

WO2016105119A1 deals with design of chopper dies as well. The shear stress during extrusion shall be reduced to effect less extractable content. However, there is no statement regarding viscoelastic behavior of the later SAP.

EP 3067370 B1 discloses a process for preparing SAP with a certain gel strength. CRC, AUL and FSR shall be well-balanced. A relationship between outlet diameter of employed extruder and gel strength is established. The number of outlets of the extruder is not given.

US 2021298962 A1 deals with production of a feminine hygiene absorbent article. In the course of this polymer gel is extruded by means of a noodle machine having a perforated plate with 12 holes having a hole diameter of 8 mm. The thickness of the perforated plate was 16 mm. The overall diameter of the hole plate is not disclosed. The ratio of internal length to internal diameter of the extruder (L/D) was 4. This value relates to the caliber of the extruder barrel of the noodle machine. However, the ratio of length and diameter of the holes are not given.

EP 2565211 A1 relates to superabsorbents based on recent carbon sources. During exemplified production hydrogel was finely crushed by a meat chopper having a hole diameter of 8 mm. Hole length and plate diameter are not given. Meat chopper's hole number is not given either.

EP 3248993 A1 discloses fast superabsorbents with low vortex values. During their preparation, hydrogel is cut into chunks using a meat chopper. Further information on meat chopper are not given.

US 2007060691 A1 mentions the use of a laboratory extruder made by MADO GmbH for braking hydrogel into small pieces. No further information on machine parameters is given.

US 2014312273 A1 aims on producing a water-absorbing polymer which has an improved swell rate and faster absorption of liquids, while simultaneously maintaining the overall quality, and more particularly a high permeability. In the course of this process, gel is comminuted with a meat grinder. Parameters of meat grinder are not disclosed.

The properties balanced in EP 3406655 A1 are T20, CRC and AUP. A chopping index is constructed by aperture ratio of employed hole plate, screw speed and solid content of extruded hydrogel.

Both EP 2944376 A1 and U.S. Ser. No. 10/130,527 B2 disclosing a process for preparing fast superabsorbents (T20=104 s) by forced agglomeration of two fractions of smaller polymeric particles. Thus, obtained superabsorbents are considered predominately as agglomerates. A structural drawback of agglomerated superabsorbents is their reduced mechanical strength: Agglomerated particles necessarily comprise grain boundaries. Such grain boundaries form breaking points within the partice. Under enhanced mechanical stress, agglomerates tend to deagglomerate by breaking off grain boundaries. Thus, superabsorbents prepared by forced agglomeration are less stable than non-agglomerated, namely monolithic superabsorbents. A further drawback of forced agglomeration as disclosed in EP 2944376 A1 and U.S. Ser. No. 10/130,527 B2 is the process-intensive method of extruding/swelling/re-extruding to create a faster product. Doing that in a plant situation would be a definite capital expense. At a glance, within present prior art there is no teaching how both absorption speed of the superabsorbent and thixotropy of the corresponding hydrogel can be positively affected.

Accordingly, object of the present invention is providing a water-absorbing polymer with improved absorption speed and less thixotropic behavior as well as a process for preparing such polymer on industrial scale.

This object has been solved by a method for preparing a water-absorbing polymer comprising the following steps:
a) providing a monomer mixture comprising the following components:
   at least one ethylenically unsaturated monomer which bears an acid group and which is optionally at least partially neutralized;
   water;
   at least one crosslinker;
   at least one initiator or at least a part of an initiator system;
   optionally at least one ethylenically unsaturated co-monomer polymerizable with above mentioned monomer;
   optionally at least one water-soluble polymer;
   optionally at least one precursor of a blowing agent;
   optionally further components;
b) providing a reactor with a reaction vessel;
c) providing an extruder having an extrusion vessel, whereby extrusion vessel is capped with a hole plate, whereby said hole plate comprises a plurality of through holes, each through hole having an equivalent diameter d and an axial length l, whereby the total void area of the hole plate established by said through holes is V and whereby the total area of the hole plate including the void area is A, whereby the geometry of the hole plate is chosen to fulfil the following requirement:

$$5.5 < 2.10686036 * {( I / d )}^{0.774457} * {( V / A )}^{- 0.117802} < 13.3$$

d) performing a polymerization within the reaction vessel by converting components of said monomer mixture to obtain a crosslinked polymer hydrogel;
e) optionally performing chemical and/or physical modifications of the hydrogel to obtain a daughter product of the hydrogel;
f) transferring said hydrogel or the daughter product thereof into the extrusion vessel;
g) optionally chopping of the hydrogel or the daughter product thereof within the extrusion vessel;
h) extruding said hydrogel or the daughter product thereof through the hole plate out of the extrusion vessel, whereby the hydrogel or the daughter product thereof is forced to pass the through holes axially to obtain an extruded hydrogel;
i) optionally performing chemical and/or physical modifications of the extruded hydrogel to obtain a daughter product of the extruded hydrogel;
k) drying extruded hydrogel or the daughter product thereof to obtain a solid polymer material;
l) grinding said solid polymer material to obtain a polymer powder;
m) sieving said polymer powder to obtain at least one sized fraction of said polymer powder;
n) post processing of said sized fraction to obtain a water-absorbing polymer whereby present step “post processing” encompasses at least one surface crosslinking step.

Such method is a first object of present invention.

The invention is based on the finding that extruding hydrogel through a hole plate obeying a complex design rule diminishes negative influence of thixotropy and increases the absorption speed of the superabsorbent made from the hydrogel.

More precisely, if replacing a conventional hole plate in a conventional SAP production process by a novel hole plate fulfilling the following requirement:

$$5.5 < 2.10686036 * {( I / d )}^{0.774457} * {( V / A )}^{- 0.117802} < 13.3$$

wherein l stands for the axial length of the holes in the hole plate,
wherein d stands for the equivalent diameter of the holes in the hole plate,
wherein V stands for the void area in the hole plate,
wherein A stands for the total area of the hole plate including the void area,
   one will obtain a water-absorbent polymer having a higher yield stress compared to a superabsorbent obtained by conventional production processes using conventional hole plates. Hence, the elasticity of inventively obtained superabsorbents is maintained up to a higher load. Thus, collapsing of elasticity due to thixotropic behavior is expected to be seen under higher load than before. The yield stress of water-absorbing polymers obtained by inventive preparation method is within range from 180 Pa to 254 Pa.

In contrast to previous teaching of extruder die geometry, present design rule incorporates two factors, namely slenderness of the holes (l/d) and void share of entire holes (V/A). Such complex specification allows properly defining stress within the hydrogel during extrusion for adjusting viscoelastic behavior quite precisely.

Present design rule has been evaluated based on a comprehensive study of different hole plates and their influence on the SAP: Hydrogel has been extruded with a multitude of hole geometries and a multitude of different hole numbers. From the hydrogel extruded with different hole plates, water-absorbent polymers have been produced and the latter have been probed regarding their performance parameters and rheology. Thus, a couple of hole plates positively influencing desired performance parameter have been found empirically.

Then, geometric characteristics of identified hole plates have been analyzed intellectually to find similarities and relevant parameters. Chosen approach was based on the principle of describing physical effects with help of dimensionless numbers following the π theorem postulated by Edgar Buckingham (1867-1940). It has been found that hole slenderness l/d and void share V/A are dimensionless numbers effecting yield stress and absorption speed most. Both numbers have been combined in form of a power function wherein each number has been equipped with a constant factor and an exponent. The numerical values of constant factors and exponents have been adjusted to the geometry of beneficial hole plates found in the laboratory with the method of least squares. Finally, quality of arrayed approximation has been validated using experimental data gained before. Suggested approximation has been found surprisingly feasible and simple.

According to the finding of present invention, yield stress and absorption speed are substantially affected by two geometric characteristics of the hole plate, namely hole slenderness l/d and void share V/A.

“Void share” is the ratio of void area V within the plate and the complete area A of the hole plate exposed to hydrogel.

The hole plate capping the extrusion vessel is always perforated. It comprises at least one orifice through which hydrogel exits extrusion vessel during extrusion. The hole plate may comprise a plurality of such holes. This should be the normal situation. The entirety of all holes is named perforation herein, regardless whether this is a single orifice or an array of many holes.

Void area is the sum of all areas of the holes through which hydrogel is extruded. (The hole plate may comprise further holes, through which hydrogel is not extruded. For example, that may be thread holes for fastening the hole plate to the extruder. As no hydrogel is extruded through such threaded holes, the latter neither affect rheology nor absorption. Accordingly, such holes are not considered when summing up void area)

The cross section of holes is not necessary constant in axial direction; the holes may be tapered as well. In that case, the area of the hole is considered at the narrows position.

The area A of the hole plate exposed to hydrogel is the area of the portion of the hole plate subjected to hydraulic pressure of the hydrogel, i.e. the portion contacting hydrogel. Portions not in contact with hydrogel, i.e. its bearing surface, are not included. However, the perforation is part of area. Hence, value of area is always greater than the value of void area. Accordingly, the number void share is always less than 1.

Present invention assumes that hole plate is plain. All areas are therefore measured simply in the plane. However, possibly the hole plate may have a three-dimensional design, for instance hemispherical. In that case, the area is calculated in the spatial area and not in its plain projection. The reason for that is that pressure profile of the hydrogel is effective spatially and not in projection.

Slenderness of the holes is the second characteristic feature of a hole plate affecting yield stress and absorption speed. Slenderness is defined as ratio of the axial length l of the holes in the hole plate and the equivalent diameter d of the holes in the hole plate. The higher the ratio l/d, the slender the holes. While in most cases slenderness will be greater than 1, stocky holes with l/d<1 are likewise possible.

If the hole extends perpendicular through the hole plate, the axial length is equal to the thickness of the hole plate.

The cross section of a hole does not necessarily need to be circular. The cross section of the hole may have other shapes as well. For characterizing the size of an orifice of any shape, equivalent diameter has been used. The equivalent diameter d of a single hole is defined as the diameter of an imaginary circle having the same area as the cross section of the actual hole. If the hole in the plate is circular, the equivalent diameter is equal to the real diameter of the hole. If the hole in the plate has a non-circular shape, the area of hole's cross section is to be measured. Then, an ideal circle is imagined having the same area then the actual bore. The calculated diameter of the ideal circle is defined as the equivalent diameter of the actual shape. For example, if the hole has a square shape with side length s, the equivalent diameter would be calculated to d=1.1288*s. If the hole would be hexagonal with an edge length of a, equivalent diameter would be calculated to d=1.8188*a

In the easiest case, equivalent diameter of all holes is equal. However, it is possible to provide a hole plate with holes of different sizes. In such case, for each individual hole the individual equivalent diameter needs to be ascertained. The equivalent diameter of the overall perforation is the arithmetic median value of all individual equivalent diameters. The same approach is applied if perforation comprises holes of different shape.

In the easiest case, the extruder comprises exactly one single hole plate. However, there are choppers known comprising a series of hole plates arrayed in the extrusion vessel downstream. In such case, only the geometry of the last hole plate actually capping the extrusion vessel is relevant for applying inventive design rule, as the pressure drop at the downstream last hole plate is the highest.

Thanks to inventive design rule, it is now possible to provide a hole plate causing a defined stress regime during extruding. Therefore, viscoelastic behavior of the superabsorbent can be controlled in the desired way by ease without further experimental trails.

A further effect of defined stress regime during extrusion is vaporizing of water trapped in polymer network due to high pressure and frictional heat. Steam generated within the hydrogel expands and causes a porous structure. The latter is conserved during drying so as the later superabsorbent obtains a porous morphology. Thanks to porosity the surface area of the superabsorbent is enlarged which finally results in higher absorption speed.

According to a preferred embodiment of the invention, the monomer mixture is made up with the following recipe:
Acrylic acid, at least partially neutralized 10 wt. % to 70 wt. %;
Water 30 wt. % to 80 wt. %;
Crosslinker: 0.05 wt. % to 5 wt. %;
Initiator 0.1 wt. % to 1 wt. %;
Co-monomer 0 wt %;
Water-soluble polymer 0 wt %;
Precursor of a blowing agent 0 wt % to 1 wt %;
Further components 0 wt. % to 20 wt. %.

All shares mentioned above are based on the total weight of the monomer mixture. The sum of all shares is 100 wt-%. A content of 0 means that related component is not added to the monomer mixture. A content ranging from 0 to a share of above 0 means that related component may be optionally included. The term “further components” covers ingredients which are neither monomer nor water nor a crosslinker nor an initiator nor a co-monomer nor a water-soluble polymer nor a precursor of a blowing agent.

This preferred recipe is well-proven for resulting in water-absorbing polymers having high yield stress, fast absorption speed and further beneficial properties.

Preferably, the monomer mixture is including a precursor of a blowing agent. The amount of the precursor shall be within the range from 1000 ppm to 1500 ppm based on the weight of the monomer mixture. Unit ppm stands for 10⁻⁶. The precursor is a substance that reacts during polymerization to produce a gas that blows the monomer mixture during polymerization to give a foam-like hydrogel. The gas is trapped in the pores of the hydrogel. Therefore, the effective blowing agent is the produced gas, not its precursor. The precursor should not react with the monomer or with co-monomer to be built into the polymer chain. It is rather intended that the precursor reacts independently from the polymerization reaction. A precursor fulfilling these requirements is selected from the group consisting of sodium carbonate, potassium carbonate, ammonium carbonate, ammonium carbamate, magnesium carbonate, calcium carbonate, barium carbonate, lithium carbonate, sodium hydrogen carbonate, potassium hydrogen carbonate, ammonium hydrogen carbonate, magnesium hydrogen carbonate, calcium hydrogen carbonate, barium hydrogen carbonate, 3-Oxopentanedioic acid, urea. The gas produced by these precursors and acting as blowing agent is in most cases carbon dioxide.

The porous structure of the hydrogel foamed by the blowing agent will be conserved in the later superabsorbent. The porosity enhances the absorption speed. Hence, the effect of vaporizing water during extrusion is improved by the blowing agent.

Within inventive method, recipe of monomer mixture includes at least one compound acting as a crosslinker. Otherwise, no three-dimensional polymer network would arise. It is possible to employ several different substances, each acting as crosslinker. The total amount of all crosslinkers within the monomer mixture is preferably greater than 10 mmol and less than 50 mmol based on 1 mol of the monomer. Unit mmol stands for 10⁻³ mol. If only one crosslinker is included, the total amount of crosslinkers is equal to the amount of the single crosslinker. A total amount of crosslinkers less than 10 mmol may result in insufficient crosslinking density that impairs gel strength. In the other way around, a total amount of crosslinkers exceeding 50 mmol may result in extraordinary gel strength. Extruding a very rigid hydrogel through inventive hole plate requires a huge amount of energy. The friction increases and the hydrogel may suffer heat damage.

The inventive method comprises a surface crosslinking step, wherein the crosslinking density of the polymer powder is increased in a surface near area of individual powder grains. Hence, the grains obtain a core/shell structure. There are several methods known in the art of superabsorbent polymer production technology to achieve such core shell structure. The surface crosslinking method fitting best to present process comprises adding at least one surface crosslinking agent and optionally at least one further component to the sized fraction of the polymer powder or to a daughter product thereof to obtain a mixture and subjecting said mixture to a thermal treatment as to increase the density of crosslinks in a surface area of the polymer powder.

Inventive process employs at least one reactor for performing polymerization and at least one extruder for extruding the hydrogel. According to a preferred embodiment of the invention, reactor and extruder are provided structurally separately. Consequently, dedicated equipment is used for performing each the polymerization step and the extruding step. Preferably, a belt reactor having an endless running belt serving as reaction vessel is used for polymerization while a meat chopper is used for performing the extruding step. Alternatively, both reaction and extrusion functionality may be performed by one combined apparatus. A well-known type of apparatus integrating both steps of reaction and extrusion is a kneader reactor.

A second object of present invention is a water-absorbing polymer having a yield stress (YS) within the range from 180 Pa to 254 Pa, whereby the water-absorbing polymer is obtained by the inventive process. Yield stress is measured according to the gel bed rheology method defined herein. It shall be pointed out that a yield stress within said range may be available with a conventional production method as well. However, if working with inventive hole plate, one will obtain a higher yield stress without changing the recipe of the monomer mixture.

The superabsorbent obtained by the inventive method has a porous structure caused by the evaporation of water during extruding. Thanks to its porosity, the absorption speed of the superabsorbent is enhanced. In particular, the water-absorbing polymer obtained by the inventive process fulfills all the following performance parameters:
a) a free swell rate (FSR) measured according to the FSR method defined herein greater than 0.48 s and less than 0.88 s.
b) an uptake of 20 g/g (T20) measured according to the k(t) method defined herein greater than 62 s and less than 125 s.

A superabsorbent achieving FSR and T20 values in that ranges are considered “superfast”.

A further characteristic of superabsorbents obtained by inventive method is a well-balanced property profile in field of gel strength and absorption speed. The gel strength is quantified by the storage modulus G′ as measured according to the gel bed rheology method disclosed herein. Beneficial property balance is exhibited by the fact that the product of storage modulus and free swell rate (G′*FSR) is greater than 2666 Pa*s and less than 5106 Pa*s

While superabsorbents prepared according to present process gain good properties in flied of speed and elasticity, other performance parameters are not negatively affected. For instance, a water-absorbing polymer obtained by the inventive method fulfills both of the following performance parameters:
a) a centrifugal retention capacity (CRC) measured according to EDANA Standard Test WSP 241.2 (5) of 25.8 g/g to 29.9 g/g
b) an absorbency under a pressure of 4.83 kPa (0.7 psi) (AUP) measured according to EDANA WSP 242.2 (5) of 23.0 g/g to 25.6 g/g

The particle size distribution of obtained water-absorbing polymers is preferable adjusted to the following particle size distribution:
600 μm to 710 μm: 5 wt %
500 μm to 600 μm 30 wt %
300 μm to 500 μm 50 wt %
150 μm to 300 μm 15 wt %.

The shares given above are target values. As reasonable deviations from said target values are acceptable, particle size distribution may be also as follows:
600 μm to 710 μm: more than 0 wt-% and less than 10 wt-%;
500 μm to 600 μm more than 20 wt-% and less than 40 wt-%;
300 μm to 500 μm more than 40 wt-% and less than 60 wt-%;
150 μm to 300 μm more than 5 wt-% and less than 25 wt-%.

In both cases, the sum of all shares within particle size distribution is 100 wt-%.

Said particle size distribution is adjusted by sieving.

Water-absorbing polymer prepared according to the invention have been subjected to a drying step and are therefore almost dry. In particular, the water trapped in the polymer network right after polymerization has been driven out during drying. Hence, after drying, material is not considered as a hydrogel any longer. Since drying to total absence of water consumes large amounts of energy, in industrial processes hydrogel is not dried to full extend. Thus, a certain moisture remains in the final product. To avoid stickiness of the final product, the moisture content needs to be capped. In a preferred embodiment the moisture content of inventive water-absorbing polymer shall be at most 10 wt-% or at most 7 wt-% or at most 5 wt-% or of at most 3 wt-%. As usual in field of superabsorbents, moisture contend is measured according to EDANA Standard NWSP 230.0.R2 (19).

It is worth to mention that water absorbing polymers obtained by the inventive process are comprising two kinds of particle types, namely monolithic particles and agglomerated particles. A monolitic particle consists of only one polymeric grain, while an agglomerated particle consist of at least two polymeric grains adhering together. Inorganic particles adhering to the polymer are not taken into consideration in this course. Thus, an agglomerate can be identified by its visible grain boundaries between polymeric grains, while a monolitic particles is free of any polymeric grain boundaries. Agglomeration of particles is a notorious phenomenon in superabsorbents. However, those skilled in the art of superabsorbents use to differentiate between forced agglomeration for avoiding smaller particles (fines) and unwanted agglomeration leading to particle oversize.

Present process is intended to avoid agglomeration. Thus, the portion of agglomerates within the inventive water absorbing particles is quite small. In particular, the ratio r of the number of monolithic particles to the number of agglomerated particles is larger than 20 or larger than 32 or larger than 50 or larger than 80 or larger than 126 or larger than 200 or larger than 500. A method to calculate ratio r is given below.

Thanks to the predominately large portion of monolithic particles, inventive superabsorbents are of proper mechanical strength.

Beyond that, inventive process is creating conditions in the extruder where speed can be achieved by extruding only once without having to go through the expense of agglomerating. Thus, present process allows preparation of fast superabsorbents with less capital expense.

A further benefit of inventive process is that extruding conditions are arranged such that shear stess applied on hydrogel during extrusion is less enough not to invoke thixotropic behavior of the later superabsorbent: Obeying inventive design rule for dimensioning hole plate geometry ensures a shear stress lower than critical shear stress causing thixotropy. Thus, water-absorbing polymer prepared by inventive process behaves (in swollen state) like a NEWTONian fluid.

As a NEWTONian fluid, superabsorbents prepared by the inventive process show a linear relationship between shear stress and strain—as long as shear stress is lower than yield stress. If stress is applied at a level exceeding yield stress, swollen superabsorbent behaves thixotropic. However, as inventive superabsorbent achieve a high yield stress, a NEwTONian behavior is to be expected during intended use in hygiene articles.

Accordingly, inventive water-absorbing polymers are characterized by a linear relationship between shear stress and strain, wherein said linear relationship is valid for shear stress values lower than yield stress. Shear stress and strain are measured according to the gel bed rheology method as defined below.

Yet another characteristic of viscoelastic matter behaving like a Newtonian fluid is derivable from diagrams displaying the relationship between storage modulus and loss modulus over strain: For non-thixotropic matter, lines of storage modulus and loss modulus do not intersect. In particular, storage modulus is larger than loss modulus. Under increasing strain, both curves approximate to each other. If strain exceeds intersection of storage modulus and loss modulus curve, thixotropic behavior is to be expected. The intersection point may be at a strain of 100% or even better at a strain of 1000%. The lower boundary of strain by which non-thixotropic can be observed is 1% or even 0.1%.

Hence, inventive water-absorbing polymers are also to be characterized by a storage modulus (G′) being larger than the loss modulus (G″) as long as water-absorbing polymer is strained in a range extending from 0.1% to 1000% or from 1% to 100%, wherein strain is measured as defined according to the gel bed rheology method defined below.

### DESCRIPTION OF OVERALL PROCESS

Present invention is now exemplified and described in detail by help of the following figures:

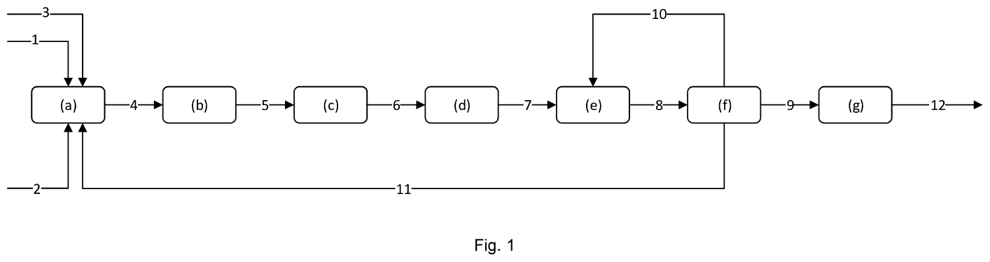

FIG. 1: Flow chart of inventive method for preparing water-absorbing polymer;

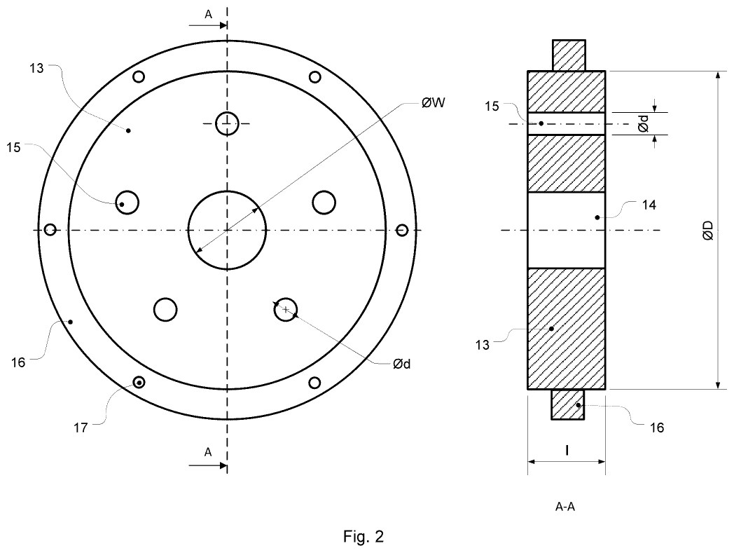

FIG. 2: Drawings of inventive hole plate, namely front view and cross section;

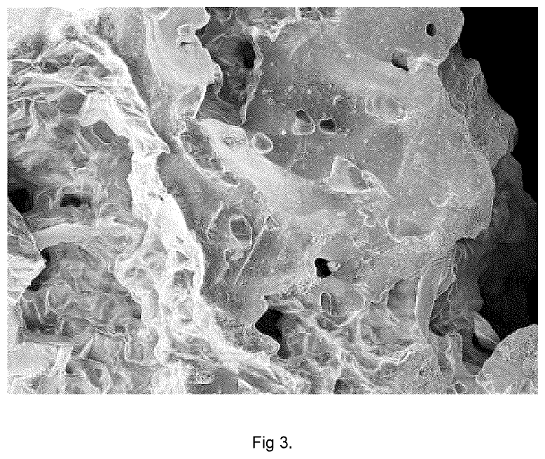

FIG. 3: SEM image of a SAP particle obtained by inventive method;

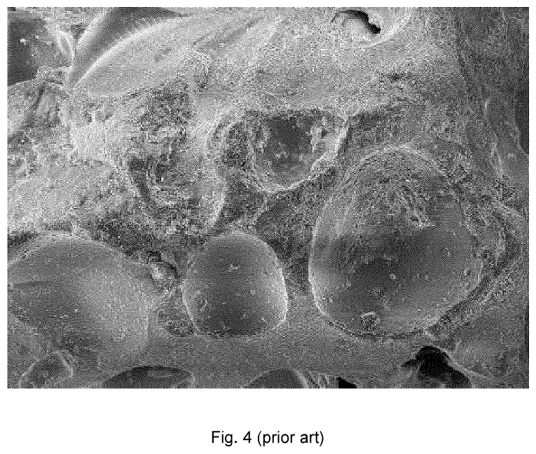

FIG. 4: SEM image of a SAP particle obtained by conventional method (prior art).

FIG. 1 depicts a process flow chart of inventive method for preparing water-absorbing polymers. It may be considered as a schematically illustration of a production plant for performing inventive method as well.

In its minimal set up, the plant comprises seven functional units (a) to (g), which are arranged to build a production strand. Each functional unit performs substantially one step of the method. A synopsis of method steps and related functional units is given in Table 1.

TABLE 1
Synopsis of method steps and functional units

| Literal | Method step | Functional unit |
|---|---|---|
| (a) | Providing monomer mixture | Receiver |
| (b) | Performing polymerization | Reactor |
| (c) | Extruding hydrogel | Extruder |
| (d) | Drying hydrogel | Dryer |
| (e) | Grinding solid polymer material | Grinder |
| (f) | Sieving polymer powder | Sieve |
| (g) | Post processing of sized fraction | Post processing section |

Basically, all functional units are provided structurally separately as dedicated equipment. However, for some functional units there is the opportunity to integrate several production steps in one multifunctional apparatus. Where this is the case, a separate note will be given. Between all functional units appropriate conveying means are arranged for accomplishing material transport from unit to unit. Conventional conveyors known in the art of producing superabsorbents are chosen. Within FIG. 1, conveying means are symbolized by arrows.

First, ethylenically unsaturated monomer 1, at least one crosslinker 2 and at least one initiator 3 are mixed within a receiver (a) to obtain a monomer mixture 4. The ethylenically unsaturated monomer 1 is dosed in the form of a liquid, namely as an aqueous monomer solution. The crosslinker 2 and the initiator 3 are dosed in form of liquid solutions as well. As water is used as solvent for the active components, monomer mixture comprises a notable amount of water.

Preferred ethylenically unsaturated monomers are α-, β-unsaturated acids, preferably α-, β-unsaturated carboxylic or sulfonic acids, including acrylic acid, methacrylic acid, crotonic acid, isocrotonic acid, itaconic acid, fumaric acid, maleic acid and 2-acrylamido-2-methyl-1-propane sulfonic acid. These acids can be used in the acidic form, but it is more preferred to use the α-, β-ethylenically unsaturated acids in their at least partially neutralized form as alkali metal salts and/or ammonium salts, including sodium and potassium salts.

Neutralization is conveniently achieved by contacting the aqueous monomer solution with an amount of base sufficient to neutralize between 10% and 95% of the acidic groups present in the acid monomers. Neutralization may be performed within the receiver 1 or beforehand. Beyond that, neutralization may be performed after polymerization, so called gel neutralization. Preferably the amount of base will be sufficient to neutralize between 40% and 85%, and most preferably between 55% and 80% of the groups present in the monomers. Suitable compounds that are useful to neutralize the acidic groups of the monomers include those bases that will sufficiently neutralize the acidic groups without having a detrimental effect on the polymerization process. Examples of such compounds include alkali metal hydroxides as well as alkali metal carbonates and bicarbonates.

Within the present process, the monomer mixture comprises at least about 10 wt.-%, more preferably at least about 25 wt.-% and even more preferably of from about 45 to about 99.9 wt.-% of α-, β-unsaturated carboxylic and/or sulfonic acids, wherein said acidic groups preferably may be present at least partly in form of a sodium salt and/or a potassium salt and/or an ammonium salt.

The acidic groups are preferably neutralized to at least about 25 mol %, more preferably to at least about 50 mol % and even more preferably of from about 50 to less than 90 mol %, more preferably from about 50 to less than 80 mol %.

The monomer mixture may comprise a mixture of said preferred monomers as well. In addition, the monomer mixture may comprise additional ethylenically unsaturated monomers in an amount of up to 60 wt.-%, including for example acryl amide, methacryl amide, maleic anhydride, alkyl esters or amides of the aforementioned monomers, including for example methyl(meth)acrylate, (meth)acrylamide, hydroxyethyl(meth)acrylate and hydroxypropyl(meth)acrylate or (meth)acrylates of polyethyleneglycol methyl ether, without being limited to these. If there are at least one additional monomer included, the such monomer is named “co-monomer” herein.

The monomeric mixture further comprises at least one crosslinker 2 for cross-linking the polymer network, i.e. a network crosslinker. Suitable crosslinkers are those which have at least two ethylenically unsaturated double bonds, those having at least one ethylenically unsaturated double bond and at least one functional group reactive towards acidic groups and those having at least two functional groups reactive towards acidic groups, or mixtures thereof.

Suitable covalent network crosslinkers include compounds having in one molecule two to four groups selected from the group consisting of CH2=CHCO—, CH2=C(CH3)CO— and CH2=CH—CH2-. Exemplary crosslinkers include diallylamine; triallylamine; diacrylates and dimethacrylates of ethylene glycol, diethylene glycol, triethylene glycol, propylene glycol, 1,4-butanediol, 1,5 pentanediol, 1,6-hexanediol, neopentyl glycol, trimethylolpropane and pentaerythritol; triacrylates and trimethacrylates of trimethylolpropane and pentaerythritol; tetraacrylate and tetramethacrylate of pentaerythritol; allyl methacrylate; tetraallyloxyethane and acrylates of the highly ethoxylated derivatives of trimethylolpropane or pentaerythritol having 3 to 30 ethylene oxide units, such as highly ethoxylated trimethylolpropane triacrylate or tetraacrylate and tetramethacrylate of pentaerythritol. Other suitable crosslinkers are monoallyl ether polyether monoacrylates such as polyethylene glycol monoallyl ether acrylate (PEG-MAE-AE). Particularly preferred are ester-type crosslinkers including highly ethoxylated trimethylolpropane triacrylate having in the range of from about 3 to about 30 EO-units per molecule (HE-TMPTA), allyl-type crosslinkers and crosslinkers carrying both acrylate and allyl-functions in the same molecule, such as for example polyethyleneglycol monoallyl ether acrylate (PEG-MAE-AE).

As a two- or multifunctional agent which is capable of forming crosslinks by reacting with the acidic groups of the polymer backbone at elevated temperatures polyethylene glycols may be employed, preferably polyethylene glycols (PEG) being liquid or pasty at room temperature (21° C. to 25° C.), such as for example PEG 600.

These network crosslinkers are distinguished from and not to be confused with the surface crosslinkers discussed below. Mixtures of the afore mentioned network crosslinkers may be employed as well.

Said network crosslinkers render the water-absorbing polymer water-insoluble, yet water-swellable. The preferred amount of crosslinker is determined by the desired degree of absorption capacity and the desired strength to retain the absorbed fluid, i.e. the desired absorption under pressure (AUP) or the absorption under load (AUL), respectively. The crosslinker advantageously is used in amounts ranging from 0.05 to 5 wt.-%, based on the total weight of the ethylenically unsaturated monomer used. More preferably the amount ranges from 0.4 wt.-% to 0.5 wt.-%. Usually if an amount of higher than about 5 wt.-% is used the polymers will have a cross-linking density that is too high and will exhibit a reduced absorption capacity. If the crosslinker is used in amounts of less than 0.05 wt.-%, the polymer usually has a cross-linking density that is too low so that when contacted with the fluid to be absorbed the polymer becomes sticky and exhibits a poor initial absorption rate.

The network crosslinker preferably may be soluble in an aqueous solution of the ethylenically unsaturated monomer, but may be merely dispersed in said solution as well, optionally in the presence of a dispersing agent. Examples of suitable dispersing agents include carboxymethyl cellulose suspending aids, methyl cellulose, hydroxypropyl cellulose and polyvinyl alcohol. Such dispersing agents are advantageously provided at a concentration between 0.0005 and 0.1 wt.-%, based on the total weight of ethylenically unsaturated monomer.

Preferably one or more of the aforementioned crosslinkers may be employed in combination with a rather small amount of at least a small amount of a polyhydric alcohol. Preferably, the monomer mixture additionally comprises at least one polyhydric alcohol as an additional crosslinker in an amount of at least 50 ppm, more preferably of from 100 to 1000 ppm, based on the total weight of ethylenically unsaturated monomer. The polyhydric alcohol preferably comprises and more preferably consists of glycerin preferably used in an amount of from 100 to 1000 ppm, based on the total weight of the ethylenically unsaturated monomer.

The monomer mixture 4 furthermore comprises at least one polymerization initiator 3.

A conventional vinyl addition polymerization initiator may be used in the polymerization of the water-soluble monomers and the crosslinker. A free-radical polymerization initiator that is sufficiently soluble in the monomer solution is preferred to initiate polymerization. For example, water-soluble persulfates such as potassium persulfate, ammonium persulfate, sodium persulfate and further alkali metal persulfates, hydrogen peroxide and water-soluble azo-compounds such as 2,2′-azobis-(2-amidinopropane) hydrochloride may be used. So-called redox initiator systems are consisting of two members, namely an oxidizing member and a reducing member. The monomer mixture may be made up with only one member of the initiator system in a first step, in a second step the second member of the initiator system is added for initiating polymerization. Hydrogen peroxide or sodium persulfate which can be used as oxidizing member of a redox initiator system can be combined with reducing member such as sulfites, amines or ascorbic acid. The amount of initiator used preferably may range of from 0.1 wt.-% to about 1 wt.-%, preferably of from about 0.1 wt.-% to about 0.5 wt.-% based on the total weight of the ethylenically unsaturated monomer.

In addition, the monomer mixture may comprise one or more chelating agents to control the rate of initiation and polymerization which otherwise may rise to an undesired level due to impurities present in the monomer mixture, such as for example heavy metal ions, in particular iron ions. The chelating agent preferably may be selected from organic polyacids, phosphoric polyacids and salts thereof. Preferably, the chelating agent may be selected from nitrilotriacetic acid, ethylene diamine tetraacetic acid, cyclohexane diamine tetraacetic acid, diethylene triamine pentaacetic acid, ethyleneglycol-bis-(aminoethylether)-N,N,N′-triacetic acid, N-(2-hydroxyethyl)-ethylene diamine-N,N,N′-triacetic acid, triethylene tetraamine hexaacetic acid, tartaric acid, citric acid, imino disuccinic acid, gluconic acid, and salts thereof. A commercially used chelating agent is the pentasodium salt of diethylene triamine pentaacetic acid.

In addition, the monomer mixture 4 may comprise water-soluble polymers such as, for example, polyvinyl alcohol, polyvinylpyrrolidone, polyglycol, polyacrylic acid, starch, starch derivatives and water-soluble or water-swellable cellulose ethers. When using polyvinyl alcohol (PVA), the latter may be partially or even fully hydrolyzed. When water-soluble polymers are employed they may preferably be used in amounts of up to about 10 wt.-%, based on the ethylenically unsaturated monomer. Such water-soluble polymers can also be used for a grafting base for the monomers to be polymerized.

Furthermore, the monomer mixture 4 may comprise recycled fines 11. As fines 11 are considered those particles produced in present process which are too small for the desired application as defined by the product specification. Polymer particles having a particle size of less than 250 μm, or preferably less than 200 μm are defined as fines in accordance with the present invention. Most fines 11 are generated by grinding (e) the solid polymer material 7 after polymerization, or by attrition of dry polymer. Said undesired product fraction is, therefore, removed from the polymer, but may be recycled by adding it to a monomer mixture 4 prior to polymerization.

Furthermore, the monomer mixture 4 may include a precursor of a blowing agent. In particular, carbonates or hydrogen carbonates are useful precursors. During polymerization such substances decompose to carbon dioxide, the actual blowing agent capable for foaming the polymerization mixture. Examples for blowing agent precursors are sodium carbonate, potassium carbonate, ammonium carbonate, ammonium carbamate, magnesium carbonate, calcium carbonate, barium carbonate, lithium carbonate, sodium hydrogen carbonate, potassium hydrogen carbonate, ammonium hydrogen carbonate, magnesium hydrogen carbonate, calcium hydrogen carbonate, barium hydrogen carbonate, 3-Oxopentanedioic acid, urea. Precursor may be added in granular form or dissolved in an aqueous solution.

Furthermore, other additives may be added to the monomer mixture. Said other additives may be selected, for example, from alkali metal chlorates, water insoluble organic or inorganic powders such as water insoluble metal oxides like silica or zinc oxide, surfactants, dispersing aids, agents to control odor, like silver salts, water soluble salts metal like aluminum sulfate or lactate, magnesium or calcium salts or further processing aids like modified non-ionic polypropylene waxes, without being limited to them.

As soon as the monomer mixture 4 is made up from components set out above, it is transferred into a reaction vessel of a reactor (b).

It shall be noted that monomer mixture 4 may be made up in the reaction vessel instead of a receiver (a). Composing monomer mixture partially within a receiver (a) and partially in the reactor (b) is also possible. In particular, the initiator 3 may be dosed to the reaction vessel. When using a redox initiator system, one member of the initiator system may be dosed to the receiver (a), while the second member is dosed to the reactor (b).

Beyond that, it is possible and even preferred to use an equipment combining both receiver (a) and reactor (b) in one apparatus. Examples for such integrated equipment are belt reactors or kneader reactors. Said reactor types are referenced in encyclopedia article cited at the beginning.

For present invention it is not relevant whether receiver (a) and reactor (b) are structurally separated or integrated. But for ease of understanding, both functional units are depicted separately here.

Within reaction vessel of reactor (b) liquid monomer mixture polymerizes to give a paste-like hydrogel 5. In the course of this reaction monomer are linked to polymer chains which are cross-linked by the crosslinker to a three-dimensional network. Water, what has initially used as solvent for the monomer, will be caged within the network. The polymer network with water trapped therein are considered as the hydrogel 5. Depending from the recipe of the monomer mixture, hydrogel 5 may contain up to 70 wt. % water. Alternatively, the hydrogel 5 may contain between 30 wt % and 70 wt % water or between 40 wt % and 60 wt % water.

Optionally, the hydrogel 5 may be subjected to a further physical and/or chemical treatment to obtain a daughter product of the hydrogel 5. Such treatment may be a neutralization, if monomer was used in acidic form. Beside that, functional additives may be dosed. For the present invention, such treatment is not relevant.

However, crucial step of inventive method is following extrusion: For this purpose, hydrogel 5 (or daughter product thereof) is feed into an extruder (c). The function of the extruder (c) is to disintegrate the hydrogel 5 into small shreds and to subject it a defined stress regime for influencing elasticity and absorption speed of the later water-absorbing polymer 12.

For fulfilling this function, extruder is provided with an extrusion vessel which is capped with a hole plate comprising a plurality of through holes. The hole plate is disclosed in detail below. The hydrogel is introduced into the extrusion vessel through an inlet. Within the extrusion vessel, there is at least one mechanical actuator applying pressure on the hydrogel towards the hole plate. As the through holes are the only outlet of the extrusion vessel in direction of the pressure, the hydrogel is forced out of the extrusion vessel through the through holes.

In the easiest case, a single worm is used as actuator. The worm extends axially through the extrusion vessel and ends up at the hole plate. The worm is rotating in a manner that its flight conveying the hydrogel towards the hole plate. The pitch of the flight may increase downstream to increase compression of hydrogel gradually. Optionally, the worm can be equipped with knifes rotating in a plane perpendicular to the axis of the worm for disintegrating the hydrogel. Such rotating knives may be situated at different positions along the axial direction of worm, for example right behind the inlet of the extrusion vessel, at the end of the extrusion vessel in front of the hole plate and even outside of the extrusion vessel behind the hole plate. A construction of an extruder having one worm and optionally rotating knives, is quite similar to meat choppers (synonym: meat mincers) used in the food industry for processing minced meat.

Alternatively, the extruder may be constructed similar to an extruder having one or two screws as they are used for compounding plastics. Such extruders are often structurally combined with kneading type reactors. In that case, the apparatus serving both as reactor and extruder comprises a single cavity integrating the function of the reaction vessel and of the extrusion vessel.

Finally, it is possible to use an apparatus similar to an injection molding machine for extruding hydrogel. The actuator for such equipment is constructed as an axially movable plunger pressing the hydrogel towards the hole plate. The plunger may be axially rotatable and equipped with a flight. Such actuator can be used as an axially movable worm, combining the cinematics of a plunger with the cinematics of a worm.

Chosen design of the actuator is not relevant for the invention, as long as the actuator is capable to apply pressure on the hydrogel towards the hole plate. As the hole plates serves as the strongest hydraulic resistor for the hydrogel on its viscoelastic flow downstream the extrusion vessel, the stress applied to the hydrogel is substantially defined by the geometry of the hole plate. The influence of the actuator is comparably low; this is the reason why the design of actuator is not that important. The high compression during extrusion affects the viscoelastic behavior of the hydrogel in a manner, that the yield stress of the later superabsorbent is enhanced. In addition to that, the water contained in the polymer network of the hydrogel is partially vaporized. The vapor exits the hydrogel into the atmosphere as soon as the hydrogel has passed the through holes. The vapor is acting as a blowing agent giving the extruded hydrogel 6 a porous structure for enhancing speed. This effect can even be observed and heard: The hydrogel exiting the orifice is notable expanding while emitting a sizzling noise.

Optionally, extruded hydrogel 6 may be subjected to additional physical and/or chemical modification to obtain a daughter product thereof.

In the next step, the extruded hydrogel 6 (or its daughter product) is fed into a dryer (d). Within the dryer (d) trapped water is driven out of the polymer network to obtain a solid polymer material 7. Drying is not done to the hilt. The remaining water content of the solid polymer material 7 exiting the dryer (d) is preferably between 4 wt. % and 7 wt. %. Dryer design has no meaning for the inventive method. For example, a circulating belt dryer can be used. Said dryer type is referenced in encyclopedia article cited at the beginning.

Then, the solid polymer material 7 withdrawn from the dyer (d) is fed into a grinder (e). Within the grinder (e) the solid polymer material 7 is ground into a polymer powder 8. The particle size distribution of the polymer powder 8 is very broad, it may contain very fine up to very coarse grains.

For employing within hygiene articles, the water-absorbing polymer should have a restricted particle size distribution, for example ranging from 150 μm to 850 μm. For bringing the polymer powder 8 into compliance with this exemplified specification, a sieve (f) is used. In its easiest set up, the sieve (f) comprises two vertically arranged horizontal extending sieve decks defining the finest and the coarsest particle size. For instance, the first sieve at the top has a mesh size so as to pass all particles below 850 μm grain size. The second sieve deck at the bottom comprises a mesh to be passed by fine particles having a grain size below 150 μm. The polymer powder 8 is fed onto the first sieve deck. From the top of the second sieve deck, a fraction 9 of the polymer powder having the desired particle size distribution ranging from 150 μm to 850 μm is withdrawn from the sieve (f). Coarse grain 10 unable to pass the first sieve is fed back into the grinder (e). Fines 11 passing the second sieve deck are recycled to the monomer mixture 4. Alternatively, fines 11 may be blended to the hydrogel 5 or may be agglomerated within a separate process (not shown).

The fraction 9 of the polymer powder having the desired particle size distribution is basically usable as an absorbent. However, such basic material fails to meet the requirements to be included in modern hygiene products as a superabsorbent. In particular, absorbency under pressure and permeability are not sufficient yet. To achieve desired performance profile, fraction 9 is transferred to a post processing section (g).

Within post processing section (g), the fraction 9 of polymer powder may be subjected several chemical and/or physical treatments to obtain a water-absorbing polymer 12 with the final properties.

Within post processing section (g) at least one surface crosslinking operation is performed. Surface crosslinking is a chemical reaction increasing the crosslinking density of the polymer in a surface-near area of the granules. Owing to this procedure, the grains obtain an egg-like core/shell structure. The core of the SAP grains retains a comparably low crosslinking density and is therefore comparably soft. The shell however achieves a higher degree of crosslinks and is therefore comparably hard. The core/shell structure of the polymer particles improves the absorbency under pressure and the permeability of the water-absorbing polymers 12.

In the surface crosslinking, the polymer particles are brought into contact with a preferably organic, chemical surface crosslinker. The surface crosslinker, especially when it is not liquid under the surface crosslinking conditions, is preferably brought into contact with the polymer particles in the form of a liquid surface crosslinking cocktail comprising the surface crosslinker and a solvent. Suitable solvents, in addition to water, are especially water-miscible, organic solvents such as, for example, methanol, ethanol, 1-propanol, 2-propanol, 1,2-propanediol, 1,3-propanediol, 1-butanol, 2-butanol, tert-butanol, iso-butanol or mixtures of organic solvents or mixtures of water with one or more of those organic solvents. The greatest preference is given to water as solvent. Within the surface crosslinking cocktail, the surface crosslinker shall be present in an amount in a range of from 5 to 75% by weight, especially from 10 to 50% by weight and more especially from 15 to 40% by weight, based on the total weight of the surface crosslinking cocktail.

The polymer particles preferably brought into contact with the liquid surface crosslinking cocktail by thorough mixing of cocktail with the polymer particles. Suitable mixing apparatus for applying the surface crosslinking cocktail onto the polymer particles being a Patterson Kelley mixer, a DRAIS turbulent mixer, a Lödige mixer, a Ruberg mixer, a screw mixer, a plate mixer or fluidised bed mixer as well as continuously operating vertical mixers in which the polymer structure is mixed at high frequency by means of rotating blades (Schugi mixer). Beyond that, mixing apparatus having heated paddles (Nara mixer) may be used.

In the surface crosslinking, the polymer particles are preferably brought into contact with at most 20% by weight, especially at most 15% by weight, more especially at most 10% by weight, very especially at most 5% by weight, of solvent. Given shares are based on the weight of the polymer particles.

Surface crosslinkers that are used in the process according to the invention are preferably understood as being compounds having at least two functional groups that are able to react with functional groups of a polymer structure in a condensation reaction (=condensation crosslinker), in an addition reaction or in a ring-opening reaction. Examples of these functional can be alcoholic, amino, aldehyde, glycidic, isocyanate, carbonate or epichloro functions. As surface crosslinkers in the process according to the invention preference is given to the following compounds: Polyols, for example ethyleneglycol, polyethyleneglycols such as diethyleneglycol, triethyleneglycol and tetraethyleneglycol, propyleneglycol, polypropyleneglycols such as dipropyleneglycol, tripropyleneglycol or tetrapropyleneglycol, 1,3-butanediol, 1,4-butanediol, 1,5-pentanediol, 2,4-pentanediol, 1,6-hexanediol, 2,5-hexanediol, glycerine, polyglycerin, trimethylolpropane, polyoxypropylene, oxyethylene-oxypropylene-block copolymer, sorbitan-fatty acid esters, polyoxyethylenesorbitan-fatty acid esters, pentaerythritol, polyvinylalcohol and sorbitol, aminoalcohols, for example ethanolamine, diethanolamine, triethanolamine or propanolamine, polyamine compounds, for example ethylenediamine, diethylenetriamine, triethylenetetraamine, tetraethylenepentaamine or pentaethylenehexaamine, polyglycidyl ether compounds such as ethyleneglycoldiglycidyl ether, polyethyleneglycoldiglycidyl ether, glycerinediglycidyl ether, glycerinepolyglycidyl ether, pentaerithritolpolyglycidyl ether, propyleneglycoldiglycidyl ether, polypropyleneglycoldiglycidyl ether, neopentylglycoldiglycidyl ether, hexanediolglycidyl ether, trimethylolpropanepolyglycidyl ether, sorbitolpolyglycidyl ether, phthalic acid diglycidyl ester, adipinic acid diglycidyl ether, 1,4-phenylenebis(2-oxazoline), glycidol, polyisocyanates, preferably diisocyanates such as 2,4-toluenediioscyanate and hexamethylenediisocyanate, polyaziridine compounds such as 2,2-bishydroxymethylbutanol-tris[3-(1-aziridinyl)propionate], 1,6-hexamethyl-enediethyleneurea and diphenylmethane-bis-4,4′-N,N′-diethyleneurea, halogen epoxides for example epichloro- and epibromohydrin and α-methylepichlorohydrin, alkylenecarbonates such as 1,3-dioxolane-2-one (ethylene carbonate), 4-methyl-1,3-dioxolane-2-one (propylene carbonate), 4,5-dimethyl-1,3-dioxolane-2-one, 4,4-dimethyl-1,3-dioxolane-2-one, 4-ethyl-1,3-dioxolane-2-one, 4-hydroxymethyl-1,3-dioxolane-2-one, 1,3-dioxane-2-one, 4-methyl-1,3-dioxane-2-one, 4,6-dimethyl-1,3-dioxane-2-one, 1,3-dioxolane-2-one, poly-1,3-dioxolane-2-on, polyquaternary amines such as condensation products from dimethylamines and epichlorohydrin. Further preferred surface crosslinkers are in addition polyoxazolines such as 1,2-ethylenebisoxazoline, crosslinkers with silane groups such as γ-glycidooxypropyltrimethoxysilane and γ-aminopropyltrimethoxysilane, oxazolidinones such as 2-oxazolidinone, bis- and poly-2-oxazolidinone and diglycolsilicates.

Of those compounds there are especially preferred as surface crosslinkers condensation crosslinkers such as, for example, diethylene glycol, triethylene glycol, polyethylene glycol, glycerol, polyglycerol, propylene glycol, diethanolamine, triethanolamine, polyoxypropylene, oxyethylene-oxypropylene block copolymers, sorbitan fatty acid esters, polyoxyethylene sorbitan fatty acid esters, trimethylolpropane, pentaerythritol, polyvinyl alcohol, sorbitol, 1,3-dioxolan-2-one (ethylene carbonate), 4-methyl-1,3-dioxolan-2-one (propylene carbonate), 4,5-dimethyl-1,3-dioxolan-2-one, 4,4-dimethyl-1,3-dioxolan-2-one, 4-ethyl-1,3-dioxolan-2-one, 4-hydroxymethyl-1,3-dioxolan-2-one, 1,3-dioxan-2-one, 4-methyl-1,3-dioxan-2-one, 4,6-dimethyl-1,3-dioxan-2-one and 1,3-dioxolan-2-one.

After the polymer particles have been brought into contact with the surface crosslinker, they are heated to a temperature in the range of from 50° C. to 300° C., preferably from 75° C. to 275° C. and especially from 150° C. to 250° C., so that, preferably with the result that, the outer region of the polymer structures is more highly crosslinked in comparison with the inner region. The duration of the heat treatment is limited by the risk of destroying the desired property profile of the polymer structures as a result of the action of heat.

Beyond surface crosslinking, the post processing section (g) may include further physical and/or chemical treatments improving the final properties of the water-absorbing polymers 12. Examples for such optional operations are rewetting or dosing of special additives. Rewetting can be done with water and/or polyethylene glycol. Examples for special additives are odor control agents, permeability boosters, anti-cracking additives, flow modification agents, surfactants, viscosity modifiers and urine stability promoters, anti-dust additives and color stabilizers.

Examples for odor control agents are cyclodextrins, zeolites, inorganic or organic salts. Zinc Peroxide is yet another odor control agent.

Further additives are Chelation agents such as ethylenediaminetetraacetic acid (EDTA); ethyleneglycoltetraacetic acid (EGTA); diethylenetriaminepentaacetic acid (DTPA); nitrile(methy-lene)triphosphonic acid; ethylenediamine-tetra(methylenephosphonic acid (EDTMP); diethylene-triamine-penta(methylenephosphonic acid (DTPMP); 1,3 diaminopropaneN,N,N′,N′,-tetraaceticacid; Na2-ethanediglycin (HEIDA); Baypure CX100; citric acid.

Further additives are mild oxidizing agents such as hydrogen peroxide, zinc peroxide; potassium peroxide; urea hydrogene peroxide; percarbonates; persulfates; Eureco LX; Eureco derivatives.

Yet another additives are pH moderating agents such as organic acids; inorganic acids; Further odor control additives: salicylic acid and salts therof; silver; zinc salts; glutamic acid, tartaric acid; benoic acid; lactic acid; cysteine hydrochloride; zeolites, cyclodextrines Natural Odor Control additives: 1,2,3,4,6-penta-O-{3,4-dihydroxy-5-[(3,4,5-trihydroxybenzoyl)oxy]benzoyl}-D-glucopyranose (Tan-nic Acid); Gallic acid; Catappa extract (Terminalia catappa), walnut leaves; whitethorn tea; Ginkgo Biloba.

Further, additives that enhance whiteness of the polymer or the long-term color stability such as against darkening, yellowing or browning may be dosed during post processing. Such additives are well known in the art and include anti-oxidants, sulfur- and phosphorous-containing compounds, chelating agents, optical brighteners and the like. Preferred additives for color stability are 2-hydroxy-2-sulfonato acetic acid, bisulfites, phosponates, ethylenediaminetetraaceticacid, ethylenediamine-N,N′-disuccinic acid, diethylenediaminepentaacetic acid, their salts and derivatives, and mixtures thereof.

Examples for permeability boosters are salts comprising a divalent or higher-valent cation of a metal and at least one organic base as anion. The divalent or higher-valent cation of a metal to be selected from the group consisting of Mg2+, Ca2+, Ba2+, Al3+, Fe2+, Fe3+, Ga3+, Ti4+, Zr4+, Cu2+ and Zn2+, greatest preference being given to Al3+. The organic base is an at least partially deprotonated mono-, di- or tri-carboxylic acid, special preference being given to deprotonated monocarboxylic acids. Also preferred are hydroxycarboxylic acids, special preference being given to at least partially deprotonated mono-, di- or hydroxy-tri-carboxylic acids, and monohydroxycarboxylic acids being especially preferred. Especially preferred anions are particularly the corresponding bases of the following acids: anisic acid, benzoic acid, formic acid, valeric acid, citric acid, glyoxylic acid, glycolic acid, glycerolphosphoric acid, glutaric acid, chloracetic acid, chloro-propionic acid, cinnamic acid, succinic acid, acetic acid, tartaric acid, lactic acid, pyruvic acid, fumaric acid, propionic acid, 3-hydroxypropionic acid, malonic acid, maleic acid, butyric acid, isobutyric acid, imidinoacetic acid, malic acid, isothionic acid, methylmaleic acid, adipic acid, itaconic acid, crotonic acid, oxalic acid, salicylic acid, gluconic acid, gallic acid, sorbic acid, gluconic acid, fatty acids, especially stearic acid and adipic acid, and β-oxybenzoic acid. Of those bases, greatest preference is given to tartrate and lactate, most preference being given to lactate. The most preferred permeability booster is aluminium lactate. Aluminium sulfate is also a preferred multivalent metal salt. The multivalent metal salt may be an aluminum sulfate such as hydrated aluminum sulfate, such as aluminum sulfate having from 12 to 14 waters of hydration. The multivalent metal salt may be aluminum lactate. Mixtures of multivalent metal salts may be employed.

It is preferable for a further anion different from the organic base to be present within the permeability booster. The anion is preferably an inorganic base. That inorganic base is preferably a deprotonated inorganic acid. As such acids there come into consideration especially the acids which are able to release two or more protons. They include especially acids containing sulphur, nitrogen or phosphorus, special preference being given to acids containing sulphur or phosphorus. Acids containing sulphur have proved especially satisfactory, especially sulphuric acid and accordingly sulphate as the salt thereof, for the base. It is preferred to use a further salt comprising aluminium sulphate as permeability booster. Preferably, at least 50% by weight, especially at least 75% by weight and more especially 100% by weight, of that salt are based on aluminium sulphate. The two different anions can be used in a ratio of from 1:100 to 100:1, preferably in a ratio of from 1:10 to 10:1 and more especially of from 1:5 to 5:1.

An additive for stabilizing color may be selected from a sulfite or bisulfite of an alkali metal, ammonium sulfite, sodium metabisulfite, ammonium bisulfite, sulfinic acid, 2-hydroxy-2-sulfinatoacetic acid, 2-hydroxy-2-sulfonatoacetic acid or sulfonic acid, and salts or derivatives of the foregoing.

Rewetting or dosing of special additives may be performed before and/or during and/or after surface crosslinking. After finishing post processing section (g), salable water-absorbing polymers 12 are obtained.

As already set out above, the geometry of the hole plate is the key for the inventive success. A simplified drawing of a hole plate 13 is shown in FIG. 2; on left-hand a front view, on right-hand a cross sectional view.

The hole plate 13 is substantially flat and circular. It is made from steel. In its center, the hole plate 13 comprises a bore 14. The bore 14 is used as a bearing for the worm of the extruder (not shown). Further, hole plate 13 comprises a plurality of through holes 15 acting as orifices for the hydrogel to exit extruder vessel which is capped by the hole plate 13. The hole plate 13 is surrounded by a flange collar 16. The flange collar 16 is designed to mount the hole plate 13 on the extruder for capping its extrusion vessel. For this purpose, flange collar 16 is provided with circularly arranged flange holes 17. Bolts (not shown) extending through said flange holes 17 are used to fasten the flange collar 16 to the extruder.

According to the present invention, hole plate 13 complies to following design rule:

$$5.5 < 2.10686036 * {( I / d )}^{0.774457} * {( V / A )}^{- 0.117802} < 13.3$$

Within this formula, l stands for the axial length of the through holes 15. As in FIG. 2 the through holes 15 extend perpendicularly to the plane of the hole plate 13 through the latter, the axial length l of the through holes 15 is identical to the thickness of the hole plate 13.

The equivalent diameter d of the through holes is the second variable value within the formula. Since hole plate 13 exemplified in FIG. 2 is equipped with through holes having a circular cross section, the equivalent diameter d of the through holes is equal to the circular diameter of the through holes. In case that through holes are non-circular or from different size, d must be calculated as set out in the general disclosure section.

Variable V stands for the void area of the hole plate. It is the sum of all cross-sectional areas of all through holes through flown by hydrogel. Since central bore 14 is not through flown by hydrogel, the cross-sectional area of bore 14 is not considered as a part of the void area. For the hole plate 13 exemplified in FIG. 2, the void area calculates V=5/4*π*d², as there are five circular through holes each having a diameter of d.

Finally, the total area A of the hole plate 13 is a variable of the design rule. The total area is the area of the face of the hole plate coming into contact with hydrogel including the void area. For the hole plate 13 exemplified in FIG. 2, the total area A calculates A=¼*π*(D−W)², with D as the diameter of the hole plate (without flange collar 16) and Was the diameter of the central bore 14. The flange collar 16 and the central bore 14 are not considered as a part of the total area, as they do not contact hydrogel.

For the hole plate 13 exemplified in FIG. 2, the term 2.10686036*(L/d)^{0.774457}*(V/A)^{-0.117802} amounts to 7.94. Hence, this hole plate 13 fulfills inventive design rule und is therefore suitable to be employed in an inventive method to produce an inventive water-absorbing polymer.

An image of an inventive water-absorbing polymer grain captured with a scanning electron microscope (SEM) at a magnification factor of 500 is reproduced in FIG. 3. The morphology is characterized by a certain porosity caused by vaporizing water during extrusion step of inventive production method. The pores are considered as a reason for enhanced absorption speed. The viscoelastic behavior is—however—not derivable from the SEM image. For assessing this, gel bed rheology measurements are required.

FIG. 4 reproduces a SEM image of a conventional SAP grain prepared according to a conventional production method (prior art). This grain is less porous and therefore not as fast as the inventive SAP grain shown in FIG. 3.

### Definition of Test Procedures

Within this disclosure, the following procedures are used for measuring product properties.

### CRC Method for Measuring Centrifugal Retention Capacity (CRC)

As defined in EDANA Standard Test WSP 241.2 (05). Said procedure is available at edana.org/publications

### AUP Method for Measuring Absorbency Under a Pressure (AUP)

As defined in EDANA Standard Test WSP 242.2 (05). Said procedure is available at edana.org/publications Pressure to be applied is amounting to 4.83 kPa (=0.7 psi)

### FSR Method for Measuring Free Swell Rate (FSR)

As defined in EP 0443627 A2, page 12, lines 24 to 44.

### K(t) Method for Measuring Uptake of 20 q/q (T20)

As defined in EP 2535697 A1, paragraphs [0027] to [0071].

### Gel Bed Rheology Method for Measuring Storage Modulus G′, Loss Modulus G″, Strain ε, Shear Stress σ and Yield Stress YS

Gel bed rheology was performed with an Anton Paar rheometer equipped with a building material cell and corresponding vane (tool).

Beside other values, rheometer is capable of measuring strain ε, storage modulus G′, loss modulus G″ and shear stess σ.

A defined particle size distribution of SAP probe was adjusted: >600 μm to 710 μm 5%, >500 μm 30%, >300 μm 50%, >150 μm 15%. Dry SAP with said particle size distribution was added to the building material cell (BMC) and mounted on the rheometer prior to swelling with saline. After adding in the SAP, the BMC was tapped in order to make an even, level bed of SAP at the bottom. The amount of SAP added was equivalent to swelling of 90% the sample's CRC value using a total of 250 g of 0.9% saline. The SAP amount was calculated as follows: if the CRC=29 g/g, SAP amount=(250 g)/(29 g/g·90%)=9.58 g.

Addition of saline to the SAP in the BMC was added via a separatory funnel to the vane shaft at a rate of approximately 10 g/s. This was critical in order to minimize disturbing the SAP bed, to reduce noise in the data, and to ensure an excess of saline upon addition (saline addition rate is greater than swell rate of SAP). The rheometer testing profiles were started as the saline was added to the vane shaft. A total of 50 minutes elapsed from saline addition to the amplitude sweep interval portion that measures yield stress.

Two different rheometer testing profiles were used between the two testing runs, but were very similar.

Interval 1: Time sweep, 0.1% strain, 1 Hz oscillation, 270 data points every 5.6 s (=25 min.).
Interval 2: Time sweep, no strain/oscillation, 270 data points every 5.6 s (=25 min.).
Interval 3: Amplitude sweep, 0.1-10,000 Pa logarithmic shear stress ramp, 150 data points every 8.6 s (=22 min.), with a test termination condition if strain is >400%.

The maximum normal force F_Nₘₐₓ and maximum storage modulus G′ can be extracted directly from the time sweeps, and are defined as their maximum obtained value during the test. The yield stress is determined by analyzing the drop in storage modulus with increasing stress. If the storage modulus drop is greater than or equal to 25 Pa (absolute) and 1% (relative), both on a consistent basis, the corresponding shear stress is taken as the yield stress (YS).

### Ratio of Monolithic Particles to Agglomerated Particles

From a bulk mass of water absorbing polymers to be probed, three random samples of each 10 g are taken, namely from different places within the bulk mass, said places distantly situated from each other in the bulk mass.

From the first sample, at least 501 particles are selected randomly. Selected particles are examined visually by means of a light microscope. For each particle, examiner decides whether particle looks either agglomerated or monolithic. The number of agglomerated particles n₁ and monolithic particles m₁ is counted and noted. The ratio r₁ of m₁ and n₁ is calculated. Procedure is repeated respectively for the second sample to calculate r₂ and for the third sample to calculate r₃.

The ratio r of monolithic particles to agglomerated particles is defined as the arithmetic average of r₁, r₂ and r₃:

$$r = ( {r}_{1} + {r}_{2} + {r}_{3} ) / 3$$

### Experimental Data

### Preparing Probes of Water Absorbing Polymers

Probes of water absorbing polymers have been prepared according to general procedure as disclosed below. So far as process parameters have been varied, varied parameter is represented by a variable named $n with n being a natural number from 1 to 12.

### General Procedure

A monomer mixture consisting from 1440 g acrylic acid, which was neutralized to an extend of 74 mol-% with sodium hydroxide solution (1848.5 g, 32% sodium hydroxide solution), $1 g deionized water, $2 g polyethylene glycol 300 diacrylate (62.1%), $3 g polyethylene glycol 450 monoallyl ether acrylate (68.6%) and $4 g of a 10% aqueous sodium carbonate solution is prepared. The monomer mixture is depleted from dissolved oxygen by degassing with nitrogen and cooled to a starting temperature amounting to approximately 4° C.

After reaching starting temperature, initiator solutions (1.35 g sodium persulfate in 10 g deionized water, 0.315 g 35% hydrogen peroxide solution in 2 g deionized water and 0.068 g ascorbic acid in 5 g deionized water) are added. An exothermic polymerization reaction occurs. The final temperature amounts to approximately 105° C.

After 20 minutes of after-reaction time, obtained hydrogel was minced in a laboratory meat mincer (Mado® Primus—motor output approximately 2.2 kW). Approximately $5% of obtained hydrogel was extruded out of the meat mincer using a circular hole plate having a multitude of similar cylindrical holes (plate diameter 80 mm, hole diameter $6 mm, hole length $7, hole number $8). After that, hydrogel extruded from the meat mincer was dried for 20 minutes at 170° C. in a laboratory belt dryer (Type FAU-2-430/200/2800/250, SO 3045; LINN HIGH TERM GmbH WERK II).

The dried polymer material was roughly crushed and afterwards grounded using a cutting mill (Retsch lab cutting mill SM 100) having a 1.5 mm whole width and sieved to a powder having a particle size ranging from 150 μm to 700 μm (d50 approximately 350 μm). From the respective samples, a defined particle size distribution was adjusted: >600 μm to 710 μm 5%, >500 μm 30%, >300 μm 50%, >150 μm 15% and afterwards the samples were homogenised for at least three minutes on a tubular overhead shaker.

Respectively, three portions of each 50 g of these mixtures were coated with a solution consisting from $9 g surface crosslinking agent $10, 1.5 g deionized water, as well as $11 g aluminium lactate (30% aqueous solution) and $12 g aluminium sulphate×14 hydrate. According to this procedure, the solution is applied to polymer material powder situated in a mixer using a syringe (0.45 mm needle). After that, the coated powder is heated for a period of 30 minutes in a dry cabinet at a temperature of 180° C. Subsequently, the three portions of surface crosslinked polymer material are merged.

### EXAMPLES

Present examples have been obtained by varying parameters $1 to $12 of aforesaid general process description according to Table 2. Varied parameters are:
$1 amount of deionized water [g]
$2 amount of crosslinker polyethylene glycol (PEG) [g]
$3 amount of crosslinker monoallyl ether acrylate (MAE-AE) [g]
$4 amount of blowing agent precursor sodium carbonate [g]
   ($4=0 in cases without blowing agent)
$5 Amount of gel obtained from recipe used for this example [%]
   ($5=50 in cases wherein from one recipe two examples have been made,
   ($5=100 in cases wherein from one recipe one example has been made)
$6 hole diameter [mm](=equivalent diameter d)
$7 hole length [mm](=axial length)
$8 amount of holes in hole plate [-](=n)
$9 amount of surface crosslinking agent [g]
$10 name of substance used as surface crosslinking agent [-]
$11 amount of permeability booster aluminium lactate used [g]
$12 amount of permeability booster aluminium sulphate 14 hydrate used [g]

### Characterization of Examples

As general procedure comprises all mandatory steps of inventive method, only geometry of hole plate is decisive for characterization of examples: If geometry parameters of a certain example fulfils the requirements of inventive design rule, examples is inventive. If not, respective example is comparative.

Based on variables $6, $7 and $8 representing equivalent diameter d, axial length/and number of holes, the term 2.10686036*(l/d)⁰⁷⁷⁴⁴⁵⁷*(V/A)^{-0.117802} has been calculated. Herein, void area V is determined by the formula n*π/4*d² while area A is determined by the formula π/4*80² mm². Thus, void share V/A calculates to n*d²/6400 mm⁻²

The calculated value of the term has been used for estimating inventive character of the respective examples. If the value of the term is greater than 5.5 and less than 13.3, the example is inventive. Otherwise, the example is comparative. Results are given in Table 3.

### Assessment of Performance Data

Performance parameters CRC, AUP, T20, FSR, YS and G′ of water absorbing polymer particles as obtained in Examples 1 to 15 have been measured according to above defined methods. The product of storage modulus and free swell rate (G′*FSR) has been calculated for each example. Results are given in Table 4.

Beside YS and G′, loss modulus G″, shear stress a and strain s of all examples have been measured with rheometer according to gel bed methodology. For each example, obtained values have been plotted in two kinds of diagrams. The first diagram shows the variation of shear stress over strain (shear stress-strain-diagram), while the second diagram shows the variation of storage modules and loss modulus over strain (moduli-strain-diagram).

Diagrams are compiled under Figure array 5. The correlations between actual Figure name, diagram type and example are derivable from Table 5.

### DISCUSSION

It has been shown that inventive examples achieve a higher yield stress than comparative examples made from the same recipe. Hence, the geometry of the hole plate actually influences the elasticity of the product positively. It is expected that superabsorbents obtained by the inventive method will bear a higher load before collapsing than superabsorbents made from the same recipe but extruded in non-inventive manner.

Beyond that, the T20 values of inventive probes are lower than comparative examples made from the same recipe. Hence, inventive geometry of the hole plate influences the absorption speed of the product.

Finally, the product of storage modulus G′ and free swell rate FSR of the inventive examples is enhanced compared to the comparative examples made from the same recipe. This indicates a better product property balance of SAP made with inventive hole plates.

These findings apply to different recipes of the monomer mixture. The influence of the hole plate geometry appears therefore decisive.

Further, diagrams showing that prepared examples behave like a NEwTONian fluid over a broad range of strain:

Materials that are NEWTONian or non-thixotropic have linear relationships between the shear stress and shear rate or strain, whereas thixotropic materials have non-linear relationships.

For instance, paper by M. Dinkgreve at al shows how the stress-strain curves for thixotropic materials are highly non-linear at low shear and strain, see FIG. 11 of Dinkgreve at al for example.
M. Dinkgreve at al.: On different ways of measuring “the” yield stress. Journal of Non-Newtonian Fluid Mechanics 238 (2016), pp. 233-241. DOI 10.1016/j.jnnfm.2016.11.001.

Material obtained by the examples does not show this behavior. All of these materials show a linear relationship between shear stress and strain.

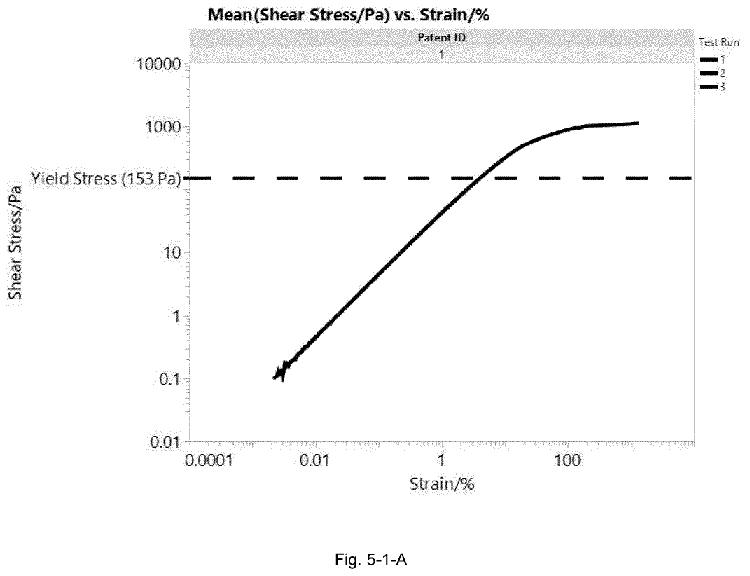

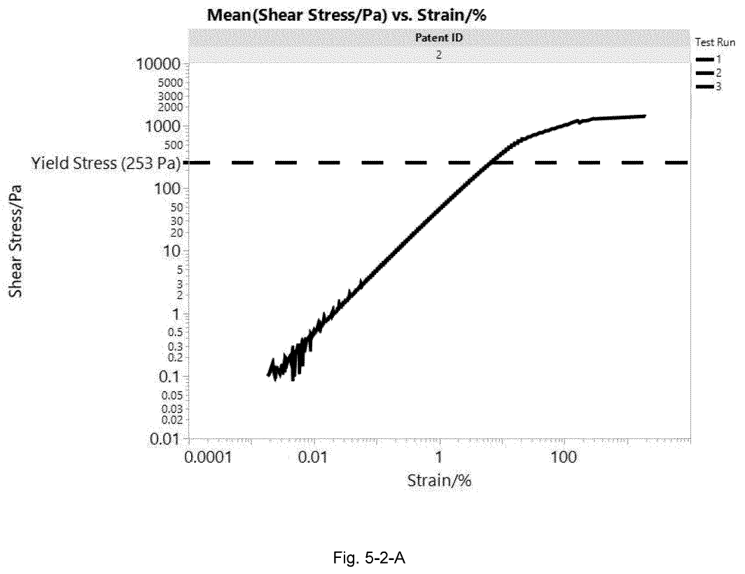

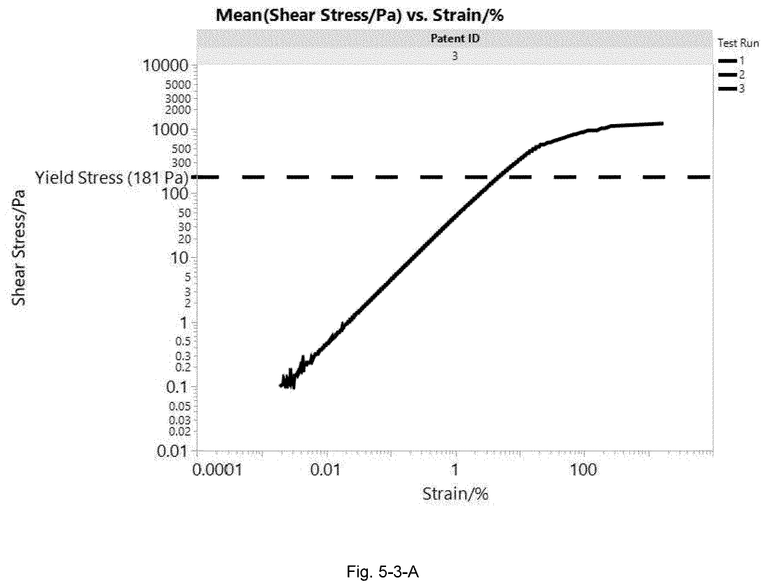

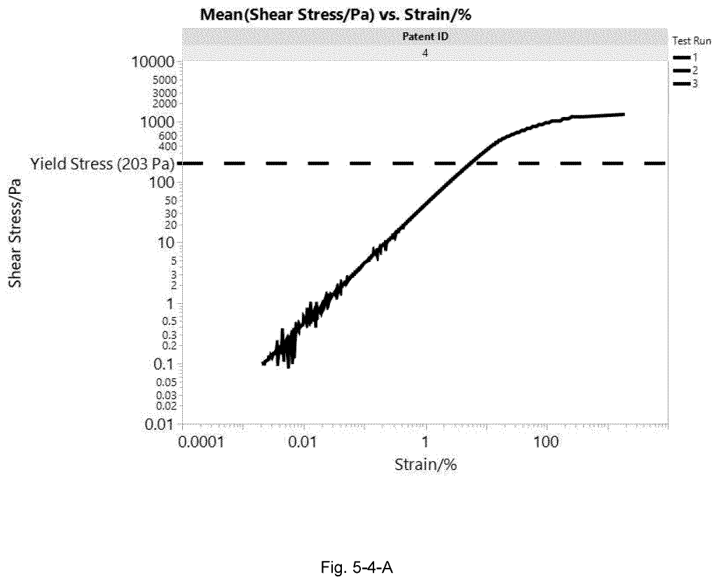

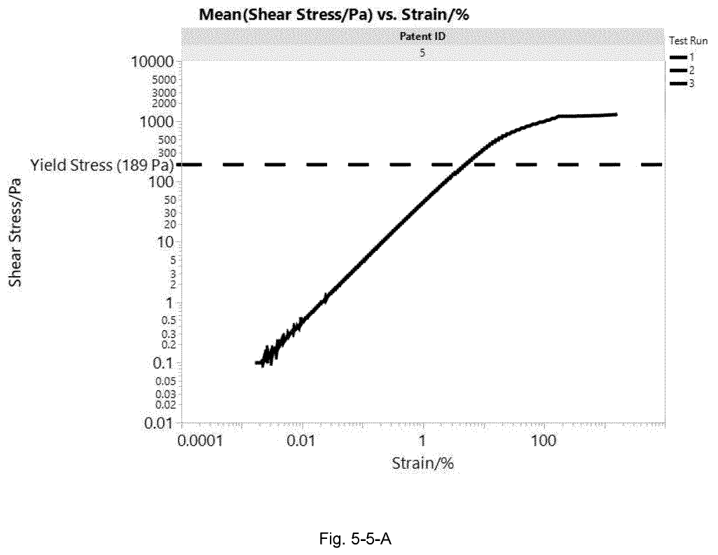

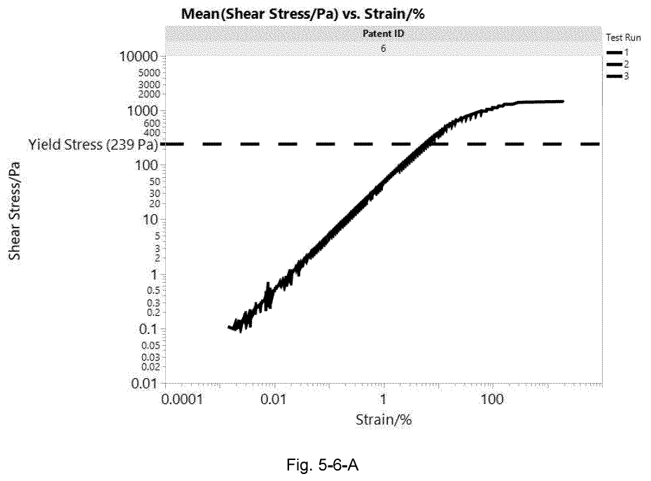

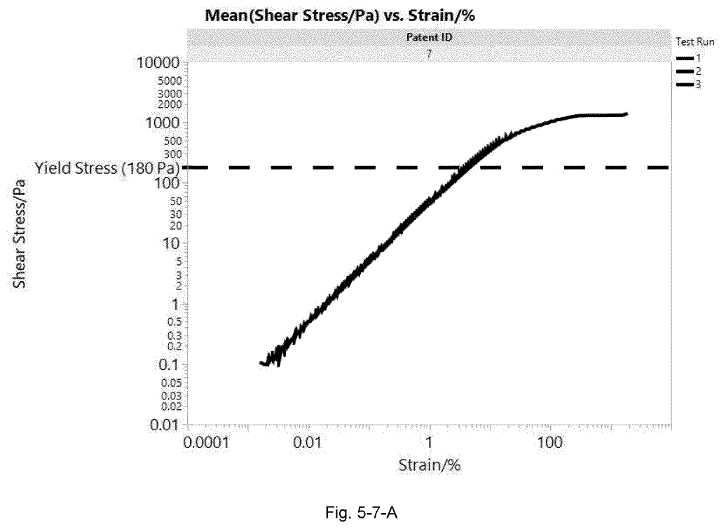

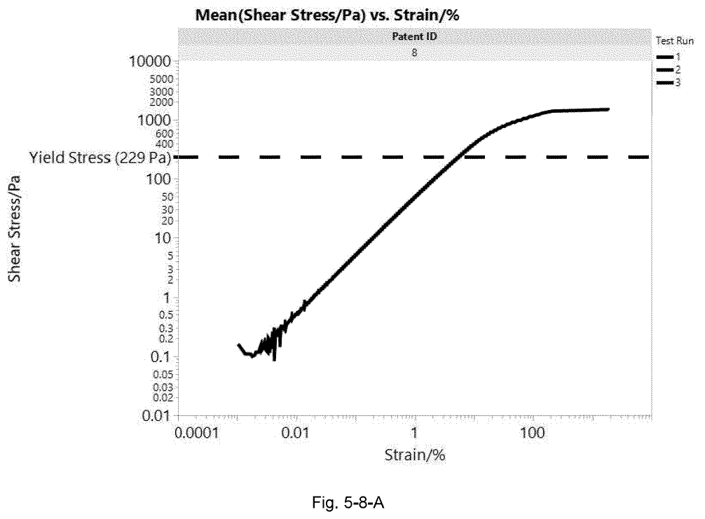

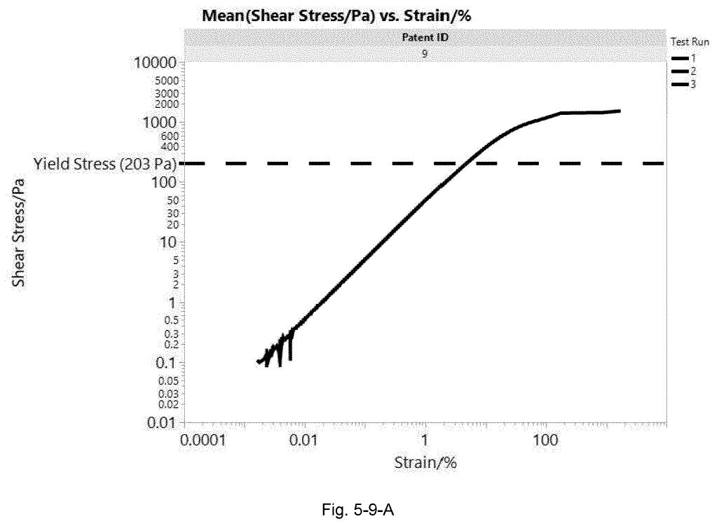

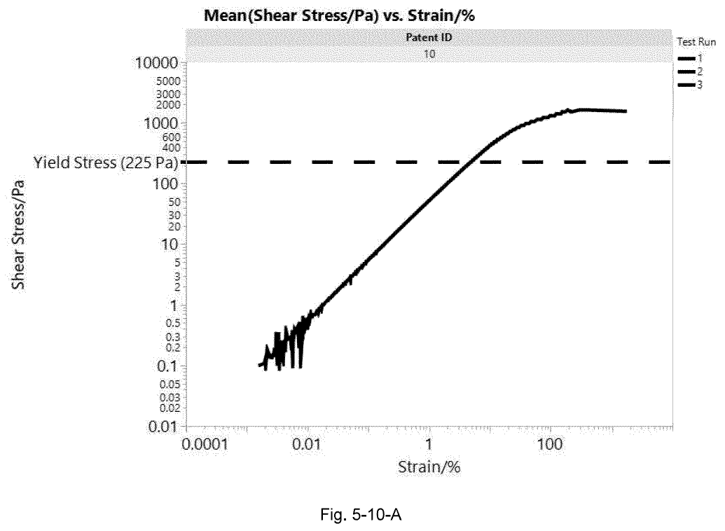

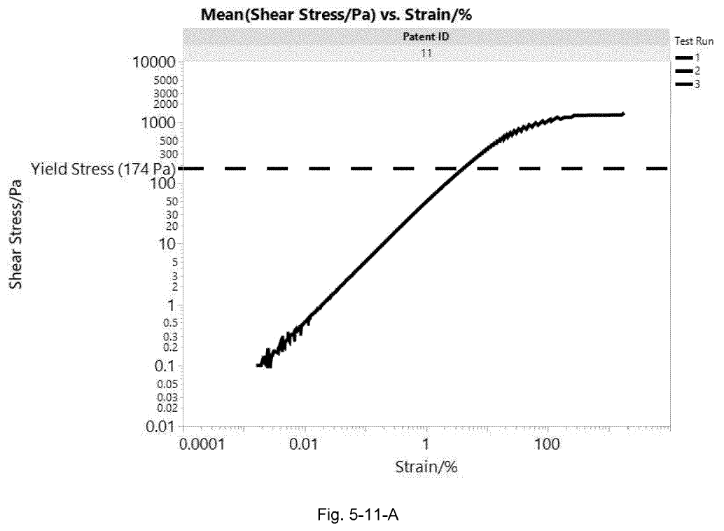

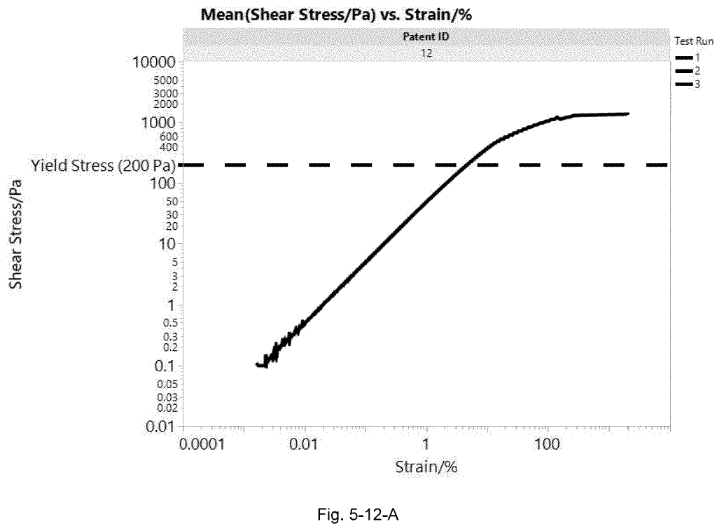

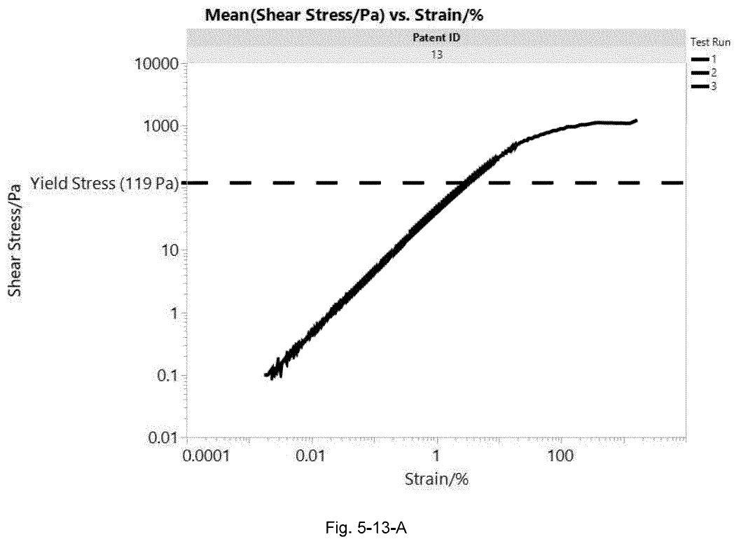

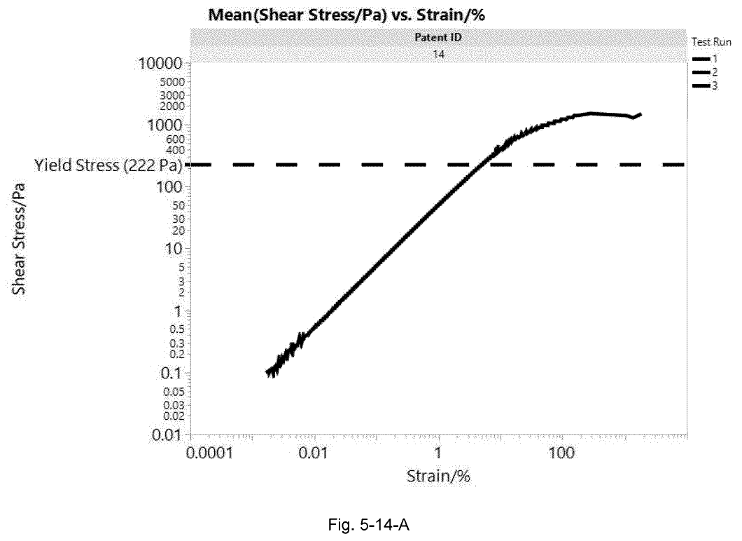

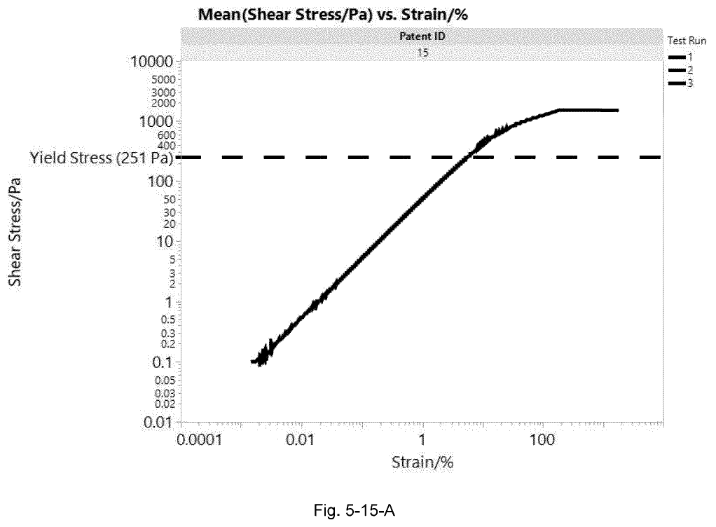

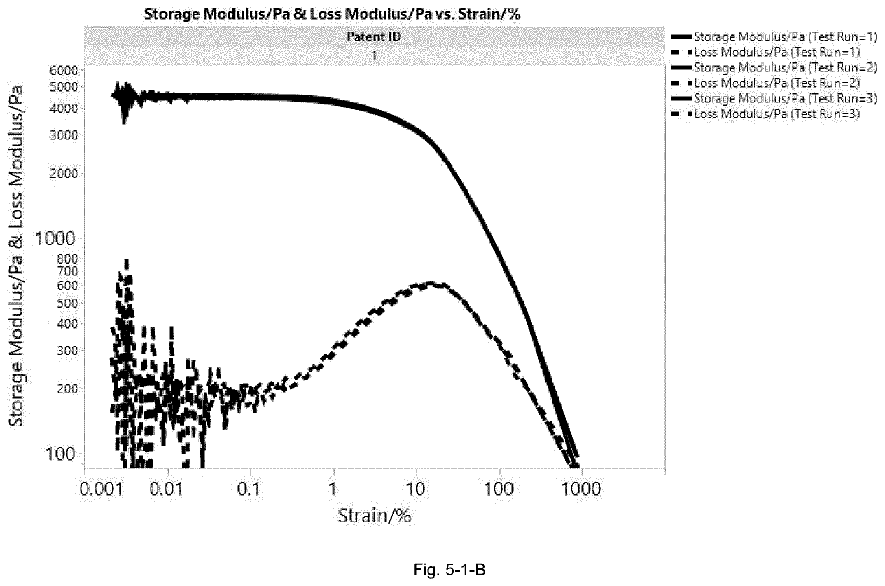

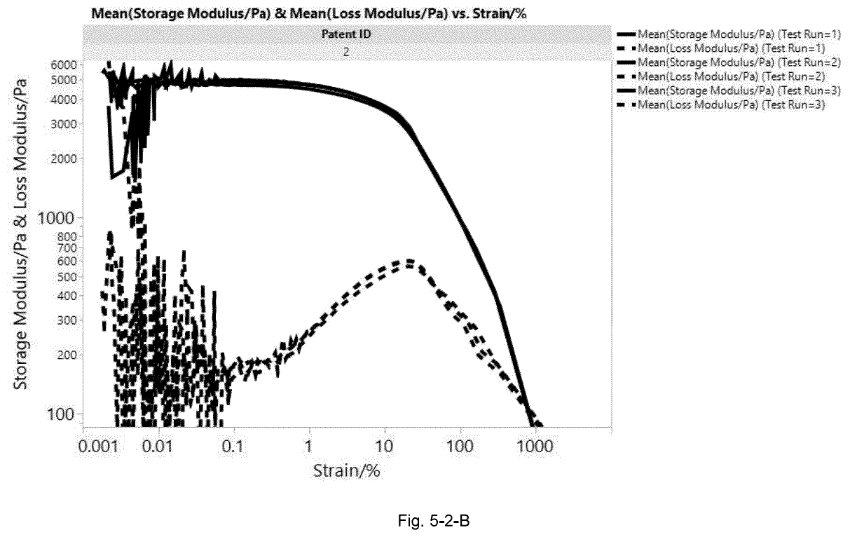

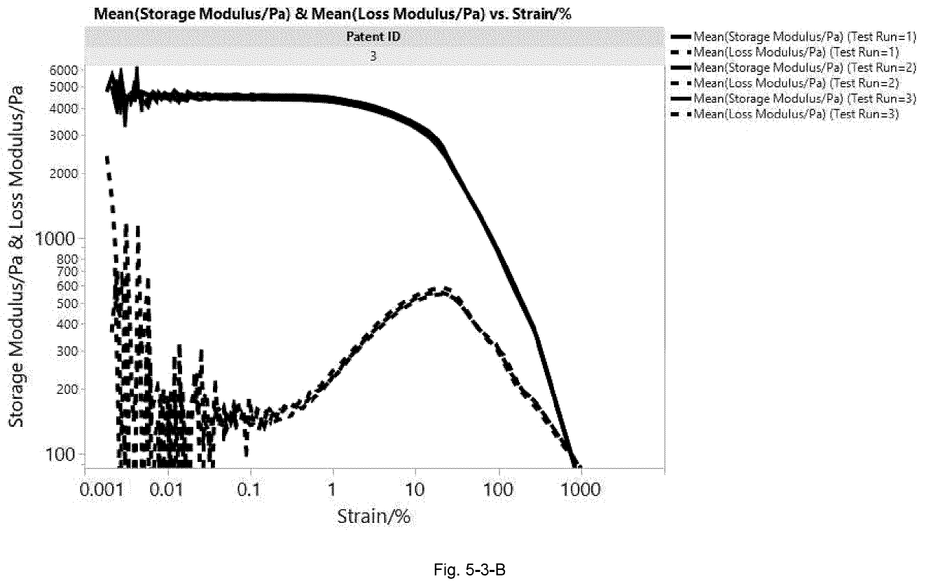

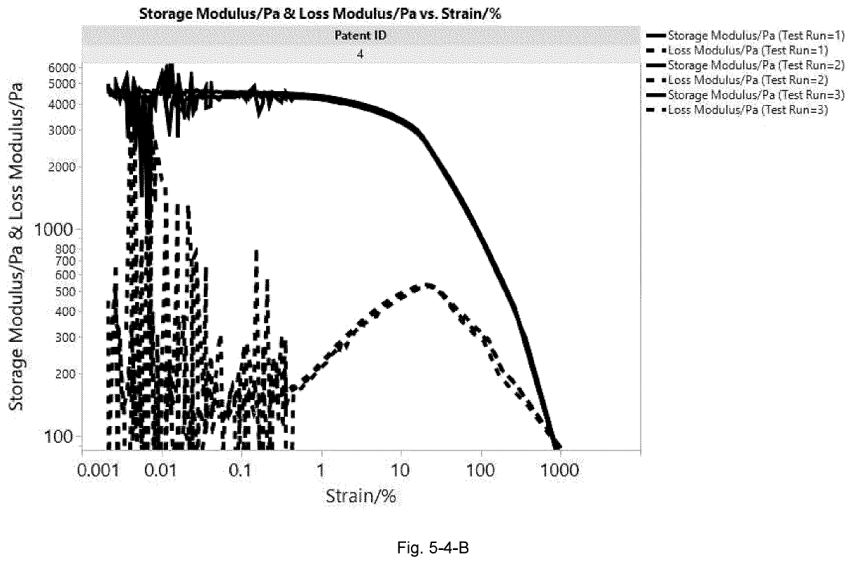

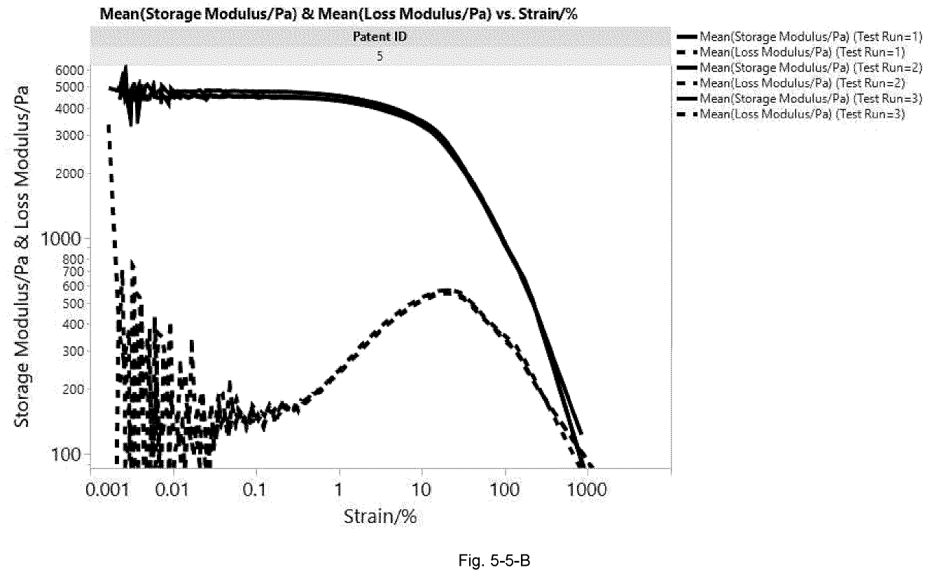

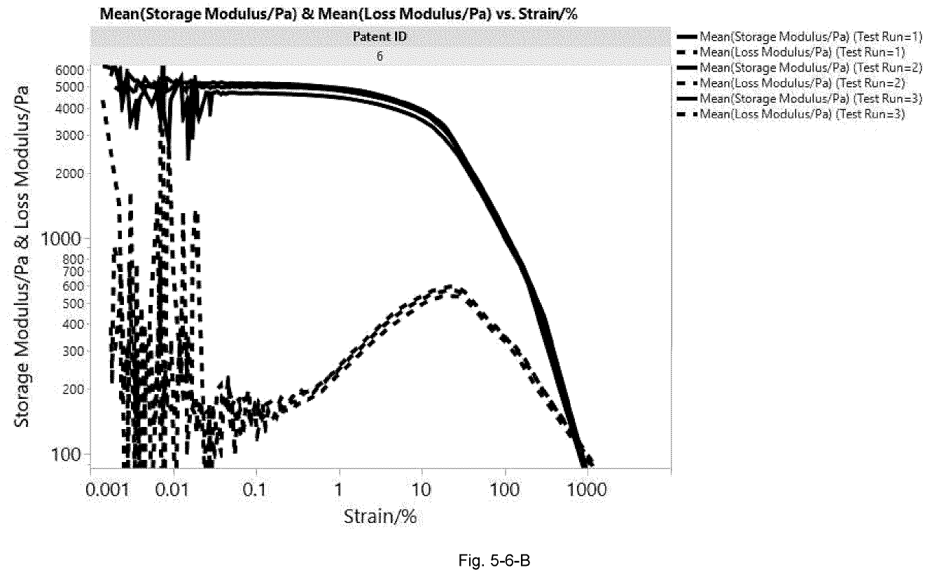

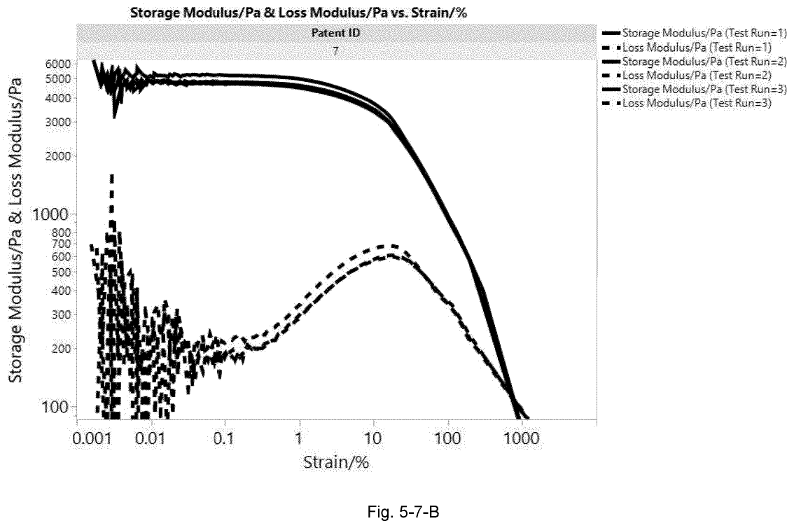

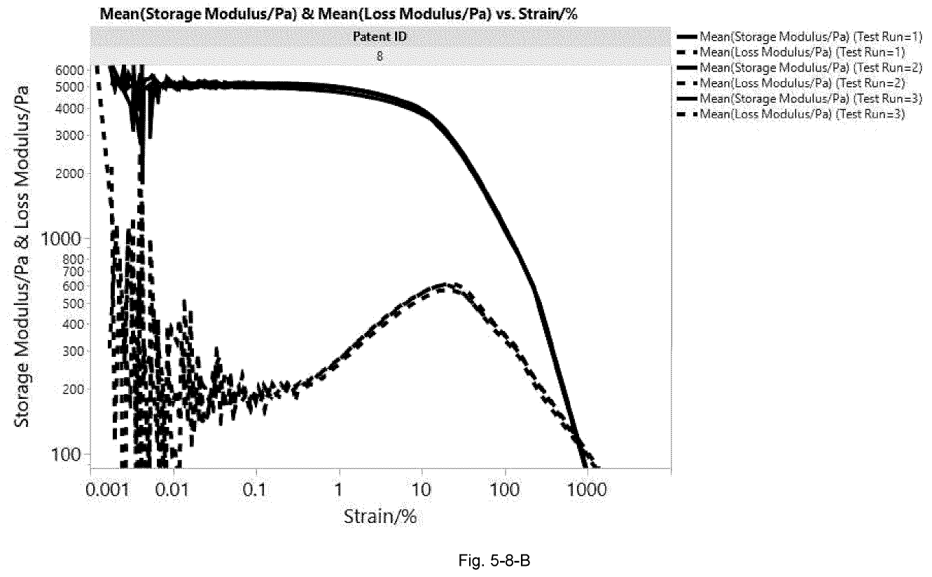

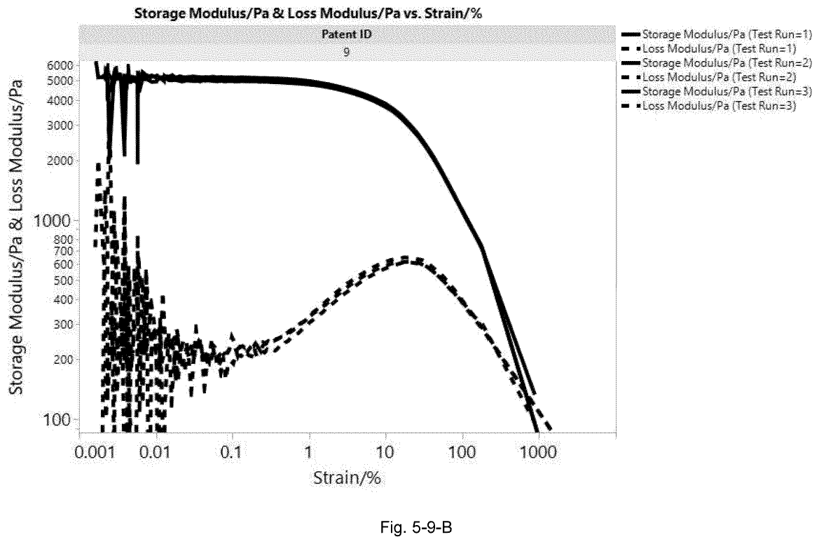

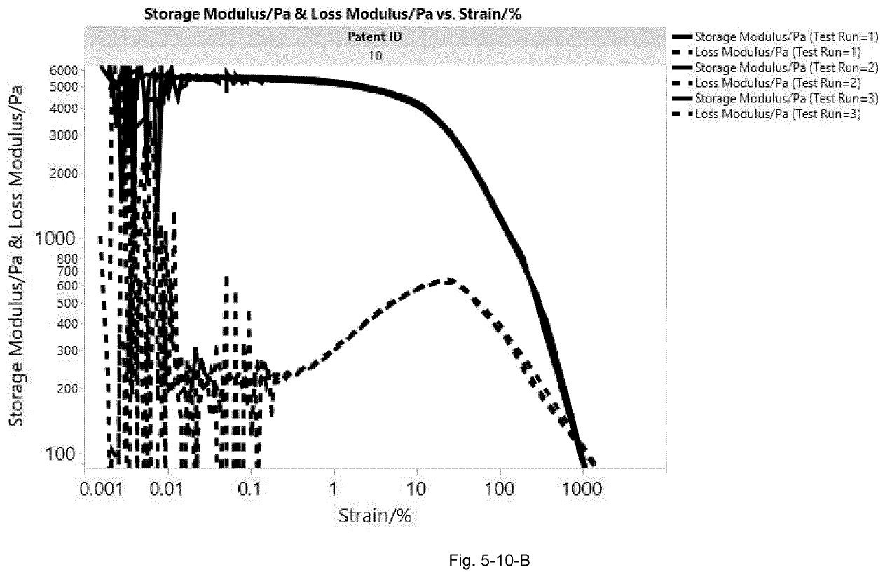

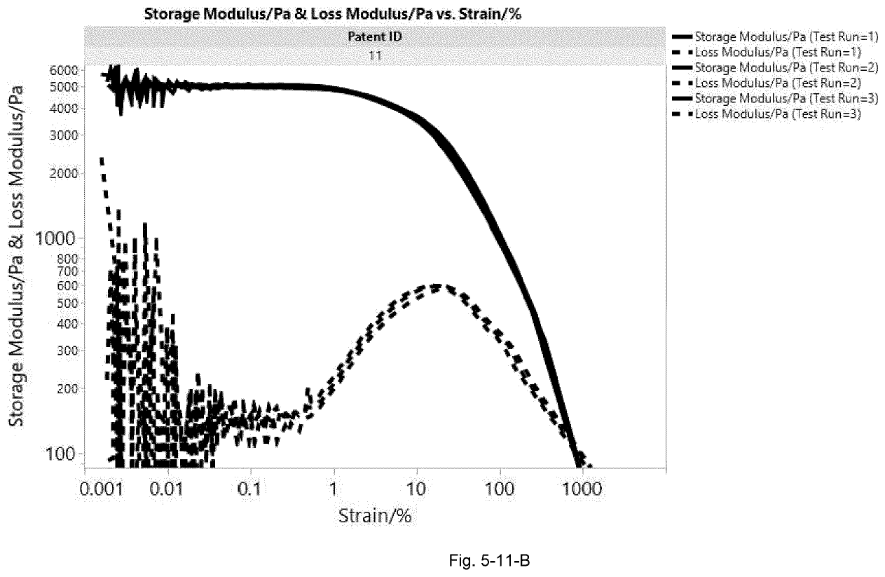

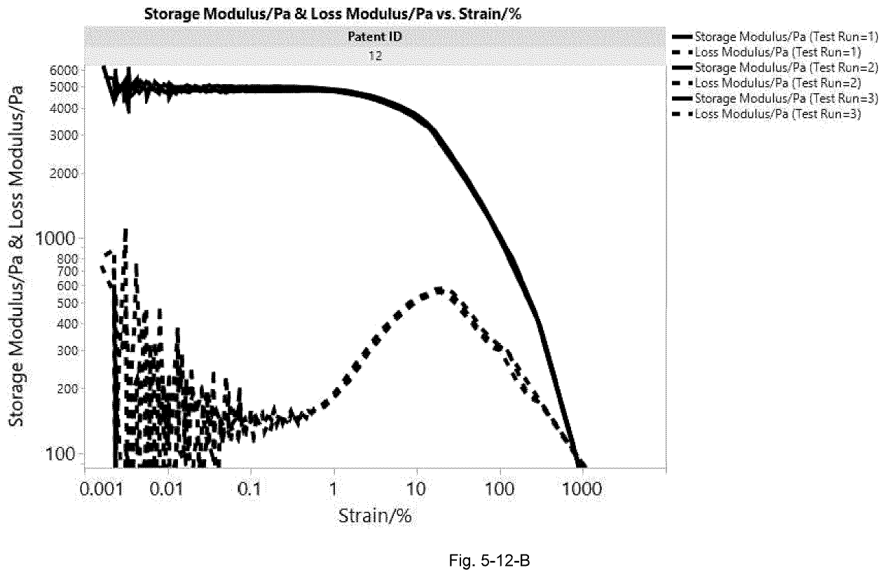

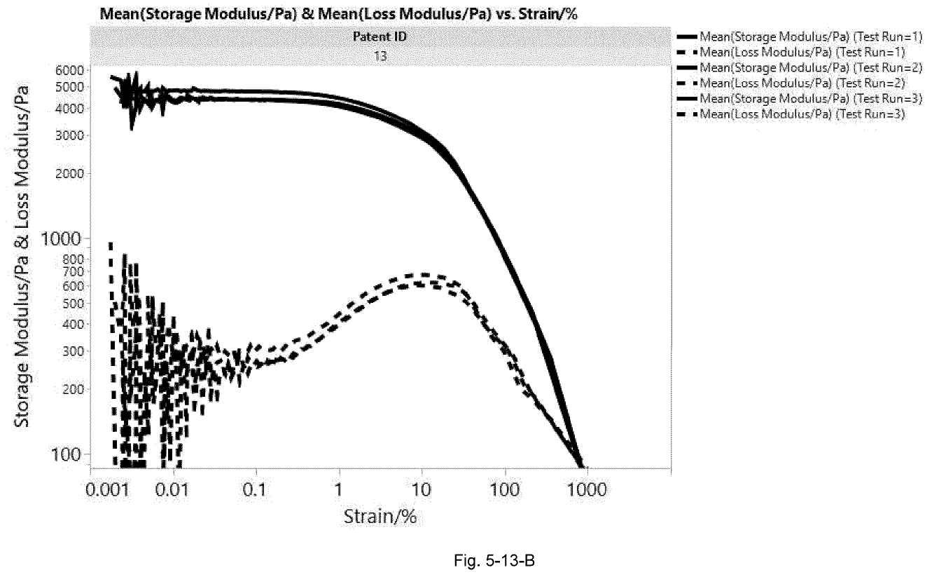

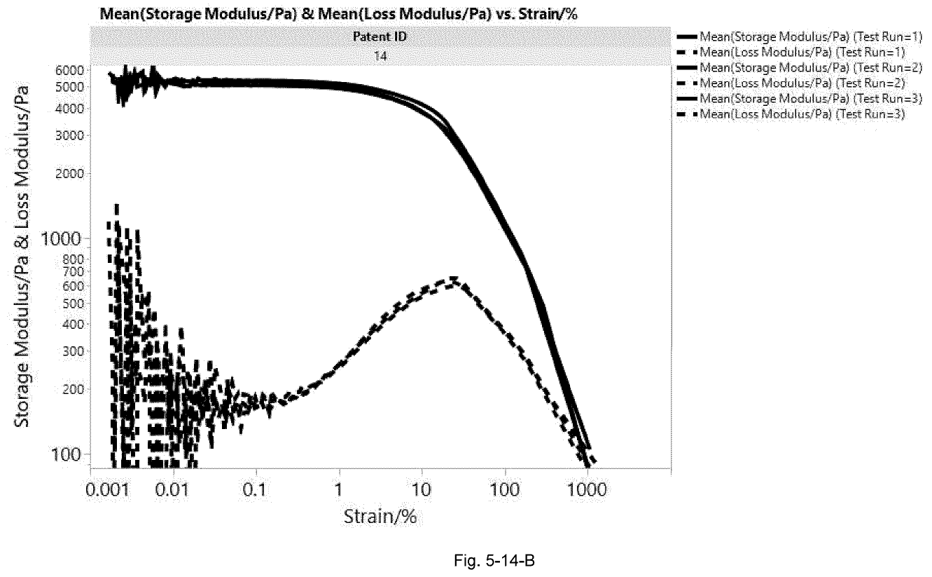

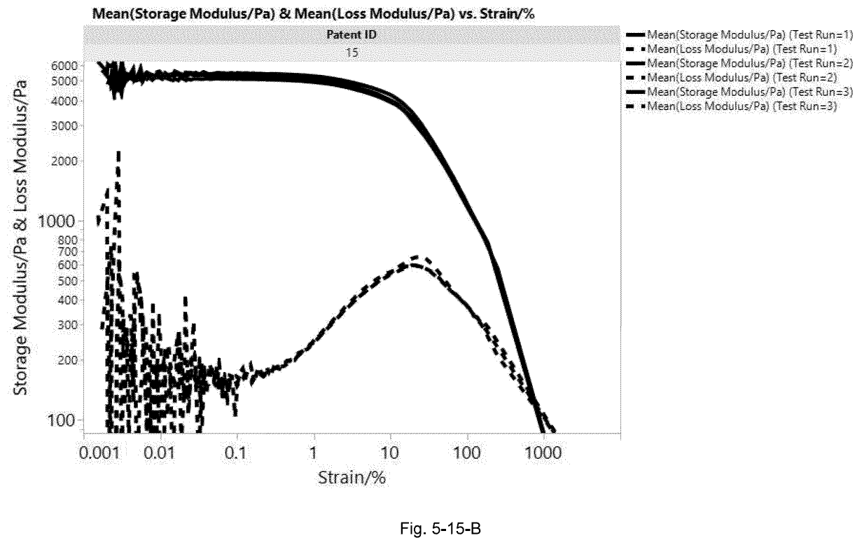

The horizontal lines in each graph FIG. 5-*-A represent the calculated yield stress for each sample test run. As can be seen in these graphs, the shear stress vs strain curves do not show thixotropic behavior in the linear region (curves below the horizontal line) and therefore application of standard definitions for yield stress are applicable. In each case, the shear stress becomes non-linear only after the yield stress (horizontal lines). After the yield stress has been surpassed, the materials no longer behave in a NEwTONian manner. As inventive examples achieve higher YS, inventive material behaves NEWTONian under higher loads than non-inventive material.

Materials prepared in the examples also do not show thixotropic behavior as evidenced by the storage (G′) and loss (G″) modulus vs strain curves. Thixotropic materials typically have storage and loss modulus curves that intersect as the material's viscosity changes with shear or stress, this is where G′=G″. This is seen in FIGS. 10a and 10b of cited paper by M. Dinkgreve at al. In those figures, the thixotropic materials all show the loss modulus intersecting the storage modulus. This is a result of the thixotropic behavior of the materials tested in the paper. The storage and loss modulus curves for material prepared in the examples do not show this behavior. The FIG. 5-*-B show the G′ and G″ curves for all 15 examples. Only at high values of strain where the test essentially is over due to “failure” of the gel is there a point where G′ and G″ might intersect. This is in contrast to behavior discussed in the paper for FIGS. 10a and 10b.

In addition, it can be seen that repeated measurements on the samples is very reproducible and consistent. There is noise in the measurements for very low values of strain, but this is typical of oscillatory measurements at low strain values. Three test runs were performed for each sample and the yield stress was determined for each test run. The values were then averaged and presented in Table 4.

Therefore, it has been proved that extruding hydrogel through a hole plate obeying disclosed design rule diminishes negative influence of thixotropy and increases the absorption speed of the superabsorbent made from the hydrogel.

TABLE 2
Value of variables according to present examples

| Example | $1 | $2 | $3 | $4 | $5 | $6 | $7 | $8 | $9 | $10 | $11 | $12 |
|---|---|---|---|---|---|---|---|---|---|---|---|---|
| 1 | 1179.5 | 7.06 | 6.3 | 0 | 50 | 5 | 8 | 100 | 0.5 | EC | 0.67 | 0.15 |
| 2 | 1179.5 | 7.06 | 6.3 | 0 | 50 | 6 | 30 | 3 | 0.5 | EC | 0.67 | 0.15 |
| 3 | 1093.3 | 4.71 | 4.2 | 90 | 50 | 5 | 8 | 100 | 0.5 | EC | 0.67 | 0.15 |
| 4 | 1093.3 | 4.71 | 4.2 | 90 | 50 | 6 | 30 | 3 | 0.5 | EC | 0.67 | 0.15 |
| 5 | 1091.7 | 5.88 | 5.3 | 90 | 50 | 5 | 8 | 100 | 0.5 | EC | 0.67 | 0.15 |
| 6 | 1091.7 | 5.88 | 5.3 | 90 | 50 | 6 | 30 | 3 | 0.5 | EC | 0.67 | 0.15 |
| 7 | 1089.5 | 7.06 | 6.3 | 90 | 50 | 5 | 8 | 100 | 0.5 | EC | 0.67 | 0.15 |
| 8 | 1089.5 | 7.06 | 6.3 | 90 | 50 | 6 | 30 | 3 | 0.5 | EC | 0.67 | 0.15 |
| 9 | 1085.0 | 9.41 | 8.4 | 90 | 50 | 5 | 8 | 100 | 0.5 | EC | 0.67 | 0.15 |
| 10 | 1085.0 | 9.41 | 8.4 | 90 | 50 | 6 | 30 | 3 | 0.5 | EC | 0.67 | 0.15 |
| 11 | 1089.5 | 7.06 | 6.3 | 90 | 50 | 5 | 8 | 100 | 0.33 | PD | 0 | 0 |
| 12 | 1089.5 | 7.06 | 6.3 | 90 | 50 | 6 | 30 | 3 | 0.33 | PD | 0 | 0 |
| 13 | 1089.5 | 7.06 | 6.3 | 90 | 100 | 6 | 8 | 72 | 0.5 | EC | 0.67 | 0.15 |
| 14 | 1089.5 | 7.06 | 6.3 | 90 | 100 | 6 | 29 | 1 | 0.5 | EC | 0.67 | 0.15 |
| 15 | 1089.5 | 7.06 | 6.3 | 90 | 100 | 4 | 20 | 3 | 0.5 | EC | 0.67 | 0.15 |

EC: ethyene carbonate
PD: 1.3-propane diol

TABLE 3
Results of calculation of character of hole plates

| Example | $6 | $7 | $8 | I/d | V/A | term | character | Example |
|---|---|---|---|---|---|---|---|---|
| 1 | 5 | 8 | 100 | 1.6 | 0.39 | 3.39 | comparative | 1 |
| 2 | 6 | 30 | 3 | 5.0 | 0.02 | 11.85 | inventive | 2 |
| 3 | 5 | 8 | 100 | 1.6 | 0.39 | 3.39 | comparative | 3 |
| 4 | 6 | 30 | 3 | 5.0 | 0.02 | 11.85 | inventive | 4 |
| 5 | 5 | 8 | 100 | 1.6 | 0.39 | 3.39 | comparative | 5 |
| 6 | 6 | 30 | 3 | 5.0 | 0.02 | 11.85 | inventive | 6 |
| 7 | 5 | 8 | 100 | 1.6 | 0.39 | 3.39 | comparative | 7 |
| 8 | 6 | 30 | 3 | 5.0 | 0.02 | 11.85 | inventive | 8 |
| 9 | 5 | 8 | 100 | 1.6 | 0.39 | 3.39 | comparative | 9 |
| 10 | 6 | 30 | 3 | 5.0 | 0.02 | 11.85 | inventive | 10 |
| 11 | 5 | 8 | 100 | 1.6 | 0.39 | 3.39 | comparative | 11 |
| 12 | 6 | 30 | 3 | 5.0 | 0.02 | 11.85 | inventive | 12 |
| 13 | 6 | 8 | 72 | 1.3 | 0.41 | 2.93 | comparative | 13 |
| 14 | 6 | 29 | 1 | 4.8 | 0.01 | 13.14 | inventive | 14 |
| 15 | 4 | 20 | 3 | 5.1 | 0.01 | 13.24 | inventive | 15 |

TABLE 4
Results of performance data assessment

| | | CRC | AUP | FSR | T20 | YS | G′ | FSR*G′ |
|---|---|---|---|---|---|---|---|---|
| Example | | [g/g] | [g/g] | [s] | [s] | [Pa] | [Pa] | [Pa*s] |
| 1 | (comp) | 28.5 | 25.0 | 0.36 | 145 | 152.50 | 4843.39 | 1743.62 |
| 2 | (inv) | 27.8 | 24.7 | 0.50 | 100 | 253.20 | 5335.61 | 2667.81 |
| 3 | (comp) | 30.6 | 26.2 | 0.48 | 111 | 181.03 | 5045.24 | 2421.72 |
| 4 | (inv) | 29.8 | 25.1 | 0.57 | 95 | 202.53 | 5142.09 | 2930.99 |
| 5 | (comp) | 29.1 | 25.6 | 0.47 | 114 | 189.00 | 5103.72 | 2398.75 |
| 6 | (inv) | 28.5 | 24.8 | 0.65 | 83 | 238.50 | 5474.89 | 3558.68 |
| 7 | (comp) | 27.2 | 24.8 | 0.47 | 116 | 179.67 | 5395.81 | 2536.03 |
| 8 | (inv) | 26.8 | 24.0 | 0.67 | 81 | 229.37 | 5591.85 | 3746.54 |
| 9 | (comp) | 26.2 | 23.9 | 0.58 | 91 | 203.37 | 5688.36 | 3299.25 |
| 10 | (inv) | 25.9 | 24.1 | 0.87 | 63 | 225.10 | 5868.05 | 5105.20 |
| 11 | (comp) | 28.5 | 25.2 | 0.53 | 117 | 174.43 | 5182.34 | 2746.64 |
| 12 | (inv) | 28.2 | 23.1 | 0.54 | 125 | 200.40 | 5036.09 | 2719.49 |
| 13 | (comp) | 28.3 | 25.5 | 0.47 | 124 | 119.37 | 4840.12 | 2274.86 |
| 14 | (inv) | 27.8 | 25.4 | 0.70 | 79 | 222.13 | 5856.76 | 4099.73 |
| 15 | (inv) | 27.8 | 25.5 | 0.68 | 81 | 251.17 | 5906.41 | 4016.36 |

(comp): comparative example
(inv): inventive example

TABLE 5
Correlation of diagrams and examples

| | Shear stress/strain | Moduli/strain |
|---|---|---|
| Example | FIG. name | FIG. name |
| 1 (comp) | FIG. 5-1-A | FIG. 5-1-B |
| 2 (inv) | FIG. 5-2-A | FIG. 5-2-B |
| 3 (comp) | FIG. 5-3-A | FIG. 5-3-B |
| 4 (inv) | FIG. 5-4-A | FIG. 5-4-B |
| 5 (comp) | FIG. 5-5-A | FIG. 5-5-B |
| 6 (inv) | FIG. 5-6-A | FIG. 5-6-B |
| 7 (comp) | FIG. 5-7-A | FIG. 5-7-B |
| 8 (inv) | FIG. 5-8-A | FIG. 5-8-B |
| 9 (comp) | FIG. 5-9-A | FIG. 5-9-B |
| 10 (inv) | FIG. 5-10-A | FIG. 5-10-B |
| 11 (comp) | FIG. 5-11-A | FIG. 5-11-B |
| 12 (inv) | FIG. 5-12-A | FIG. 5-12-B |
| 13 (comp) | FIG. 5-13-A | FIG. 5-13-B |
| 14 (inv) | FIG. 5-14-A | FIG. 5-14-B |
| 15 (inv) | FIG. 5-15-A | FIG. 5-15-B |

### REFERENCES

a Receiver
b Reactor
c Extruder
d Dryer
e Grinder
f Sieve
g Post processing section
1 Monomer
2 Crosslinker
3 Initiator
4 Monomer mixture
5 Hydrogel
6 Extruded hydrogel
7 Solid polymer material
8 Polymer powder
9 Fraction of polymer powder
10 Coarse grain
11 Fines
12 Water-absorbing polymer
13 Hole plate
14 Bore
15 Through hole
16 Flange collar
17 Flange hole
d Equivalent diameter of through hole
l Axial length of through hole
D Diameter of hole plate
W Bore diameter

What is claimed is:

1. A method for preparing a water-absorbing polymer comprising the following steps:
a) providing a monomer mixture comprising the following components:
at least one ethylenically unsaturated monomer which bears an acid group and which is optionally at least partially neutralized;
water;
at least one crosslinker;
at least one initiator or at least one member of an initiator system;
optionally at least one ethylenically unsaturated co-monomer polymerizable with above mentioned monomer;
optionally at least one water-soluble polymer;
optionally at least one precursor of a blowing agent;
optionally further components;
b) providing a reactor having a reaction vessel;
c) providing an extruder having an extrusion vessel, whereby extrusion vessel is capped with a hole plate, whereby said hole plate comprises a plurality of through holes, each through hole having an equivalent diameter d and an axial length l, whereby the total void area of the hole plate established by said through holes is V and whereby the total area of the hole plate including the void area is A;
d) performing a polymerization within the reaction vessel by converting components of said monomer mixture to obtain a crosslinked polymer hydrogel;
e) optionally performing chemical and/or physical modifications of the hydrogel to obtain a daughter product of the hydrogel;
f) transferring said hydrogel or the daughter product thereof into the extrusion vessel;
g) optionally chopping of the hydrogel or of the daughter product thereof within the extrusion vessel;
h) extruding said hydrogel or the daughter product thereof through the hole plate out of the extrusion vessel, whereby the hydrogel or the daughter product thereof is forced to pass the through holes axially to obtain an extruded hydrogel;
i) optionally performing chemical and/or physical modifications of the extruded hydrogel to obtain a daughter product of the extruded hydrogel;
k) drying extruded hydrogel or the daughter product thereof to obtain a solid polymer material;
l) grinding said solid polymer material to obtain a polymer powder;
m) sieving said polymer powder to obtain at least one sized fraction of said polymer powder;
n) post processing of said sized fraction to obtain a water-absorbing polymer, whereby present step “post processing” encompasses at least one surface crosslinking step;
wherein
the geometry of the hole plate is chosen to fulfil the following requirement:
$$5.5 < 2.10686036 * {( I / d )}^{0.774457} * {( V / A )}^{- 0.117802} < 13.3$$

2. A method according to claim 1, wherein the monomer mixture is provided according the following recipe, wherein given shares sum up to 100 wt.-% and wherein given shares are based on the total weight of the monomer mixture:
Acrylic acid, at least partially neutralized 10 wt. % to 70 wt. %;
Water 30 wt. % to 80 wt. %;
Crosslinker 0.05 wt. % to 5 wt. %;
Initiator 0.1 wt. % to 1 wt. %;
Co-monomer 0 wt. %;
Water-soluble polymer 0 wt. %;
Precursor of a blowing agent 0 wt. % to 1 wt. %;
Further components 0 wt. % to 20 wt. %.

3. A method according to claim 2, wherein the monomer mixture comprises a precursor of a blowing agent, wherein the amount of the precursor is within the range from 1000 ppm to 1500 ppm based on the weight of the monomer mixture and wherein the precursor is at least one member selected from the group consisting of sodium carbonate, potassium carbonate, ammonium carbonate, ammonium carbamate, magnesium carbonate, calcium carbonate, barium carbonate, lithium carbonate, sodium hydrogen carbonate, potassium hydrogen carbonate, ammonium hydrogen carbonate, magnesium hydrogen carbonate, calcium hydrogen carbonate, barium hydrogen carbonate, 3-Oxopentanedioic acid, urea.

4. A method according to claim 2, wherein the total amount of crosslinkers within the monomer mixture is greater than 10 mmol and less than 50 mmol based on 1 mol of the monomer.

5. A method according to claim 1, wherein the surface crosslinking step comprises adding at least one surface crosslinking agent and optionally at least one further component to the sized fraction of the polymer powder or to a daughter product thereof to obtain a mixture and subjecting said mixture to a thermal treatment as to increase the density of crosslinks in a surface area of the polymer powder.

6. A method according to claim 1, wherein the reactor and extruder are provided structurally separately.

7. A water-absorbing polymer having a yield stress (YS) within the range from 180 Pa to 254 Pa, wherein the yield stress is measured according to the gel bed rheology method defined herein, wherein the water-absorbing polymer is obtained by a method according to claim 1.

8. A water-absorbing polymer according to claim 7, wherein the water-absorbing polymer cumulatively fulfills all the following performance parameters:
a) a free swell rate (FSR) measured according to the FSR method defined herein greater than 0.48 s and less than 0.88 s.
b) an uptake of 20 g/g (T20) measured according to the k(t) method defined herein greater than 62 s and less than 125 s.

9. A water-absorbing polymer according to claim 8, wherein the water-absorbing polymer having a storage modulus (G′) measured according to the gel bed rheology method defined herein, characterized in that the product of storage modulus and free swell rate (G′*FSR) is greater than 2666 Pa*s and less than 5106 Pa*s.

10. A water-absorbing polymer according to claim 8, wherein the water-absorbing polymer cumulatively fulfills the following performance parameters:
a) a centrifugal retention capacity (CRC) measured according to EDANA Standard Test WSP 241.2 (5) of 25.8 g/g to 29.9 g/g
b) an absorbency under a pressure of 4.83 kPa (AUP) measured according to EDANA WSP 242.2 (5) of 23.0 g/g to 25.6 g/g.

11. A water-absorbing polymer according to claim 7, wherein the following particle size distribution adjusted by sieving:
600 μm to 710 μm: more than 0 wt-% and less than 10 wt-%, target 5 wt-%;
500 μm to 600 μm more than 20 wt-% and less than 40 wt-%, target 30 wt-%;
300 μm to 500 μm more than 40 wt-% and less than 60 wt-%, target 50 wt-%;
150 μm to 300 μm more than 5 wt-% and less than 25 wt-%, target 15 wt-%;
wherein the sum of all shares is 100 wt-%.

12. A water-absorbing polymer according to claim 7, wherein a moisture content according to EDANA Standard NWSP 230.0.R2 (19) of at most 10%-wt or of at most 7 wt-%, of at most 5 wt-% or of at most 3 wt-%.

13. A water-absorbing polymer according to claim 7, wherein water absorbing polymer comprises monolithic particles and agglomerated particles, characterized in that the ratio r of the number of monolithic particles to the number of agglomerated particles is larger than 20 or larger than 32 or larger than 50 or larger than 80 or larger than 126 or larger than 200 or larger than 500, whereby the ratio is to be measured as defined in the description.

14. A water-absorbing polymer according to claim 7, wherein a linear relationship between shear stress (s) and strain (e), wherein said linear relationship is valid for shear stess values (s) lower than yield stress (YS) and wherein shear stress (s) and strain (e) are measured as defined in the description.

15. A water-absorbing polymer according to claim 7, wherein water-absorbing polymer having a storage modulus (G′) and a loss modulus (G″), both measured according to the gel bed rheology method defined herein, characterized in that storage modulus (G′) is larger than the loss modules (G″) as long as water-absorbing polymer is strained in a range extending from 0.1% to 1000% or from 1% to 100%, wherein strain (e) is measured as defined in the description.
